(19) United States
(12) Patent Application Publication
(10) Pub. No.: US 2018/0092909 A1
(43) Pub. Date: Apr. 5, 2018

(54) LURASIDONE NOVEL DOSAGE REGIMENS AND USE THEREOF FOR THE TREATMENT, PREVENTION, AND/OR MANAGEMENT OF AT LEAST ONE CNS DISORDER

(71) Applicant: SUMITOMO DAINIPPON PHARMA CO., LTD., OSAKA-SHI (JP)

(72) Inventor: Antony D. Loebel, Larchmont, NY (US)

(73) Assignee: SUMITOMO DAINIPPON PHARMA CO., LTD., OSAKA-SHI (JP)

(21) Appl. No.: 15/700,692

(22) Filed: Sep. 11, 2017

Related U.S. Application Data
(63) Continuation of application No. 14/361,536, filed on Feb. 19, 2015.
(60) Provisional application No. 61/589,479, filed on Jan. 23, 2012.
(60) Provisional application No. 61/566,379, filed on Dec. 2, 2011.

(51) Int. Cl. A61K 31/496; B65D 77/08

(52) U.S. Cl. CPC A61K 31/496; B65D 77/08

(57) ABSTRACT

Dosage regimens for at least one compound chosen from lurasidone and pharmaceutically acceptable salts, solvates, clathrates, and stereoisomers thereof are disclosed as are kits and methods for treatment and/or prevention of at least one CNS disorder such as, for example, mixed depression and bipolar disorder, and management of at least one CNS disorder, such as improving quality of life and reversing impairment in learning and memory associated with schizophrenia, comprising administering to a patient a therapeutically or prophylacticaily effective amount of at least one compound chosen from lurasidone and pharmaceutically acceptable salts, solvates, clathrates, and stereoisomers thereof.

This is a continuation of U.S. Ser. No. 14/361,536, filed Feb. 19, 2015, which is a national stage of International Application No. PCT/IB2012/002904, filed Nov. 30, 2012, which claims the benefits of priority to U.S. Provisional Application No. 61/566,379, filed Dec. 2, 2011, and to U.S. Provisional Application No. 61/589,479, filed Jan. 23, 2012. The entire disclosures of all of the above applications are incorporated by reference herein.

Dosage regimens for at least one compound chosen from lurasidone and pharmaceutically acceptable salts, solvates, clathrates, and stereoisomers thereof are disclosed as are kits and methods for treatment and/or prevention of at least one CNS disorder such as, for example, mixed depression, schizophrenia, and bipolar disorder, and management of at least one CNS disorder, such as improving quality of life and reversing impairment in learning and memory associated with schizophrenia, comprising administering to a patient a therapeutically or prophylactically effective amount of the at least one compound.

Lurasidone is a compound exhibiting a pharmacological activity as a psychotropic agent. Lurasidone has a chemical name (3aR,4S,7R,7aS)-2-{(1R,2R)-2-[4-(1,2-benzisothiazol-3-yl)piperazin-1-ylmethyl]cyclohexylmethyl}hexahydro-4,7-methano-2H-isoindole-1,3-dione], and has the following formula:

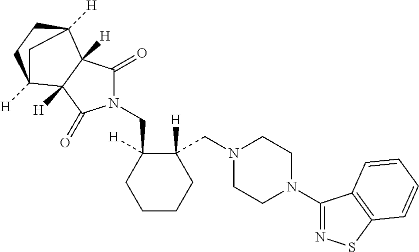 [SMILES: O=C1[C@@H]2[C@H]3CC[C@H](C3)[C@@H]2C(=O)N1C[C@@H]1CCCC[C@H]1CN1CCN(c2nsc3ccccc23)CC1]

Lurasidone is reported to have a high affinity for dopamine D₂, serotonin 5-HT_{1A}, 5-HT_{2A}, 5-HT₇, and noradrenaline α_{2c} receptors, moderate affinity for 5-HT_{1A} receptors, and minimal to no affinity for histamine H₁ and muscarinic M₁ receptors. Data from several placebo-controlled trials has demonstrated that lurasidone is effective in ameliorating the positive and negative symptoms of schizophrenia. Data from clinical and pre-clinical studies have suggested that lurasidone also demonstrates antidepressant- or anxiolytic-like effects, as well as pro-cognitive effects with potentially-reduced liability for extrapyramidal, weight and metabolic parameters, and other CNS depressant side effects.

It has been reported that patients with schizophrenia are at an increased risk of developing metabolic syndrome independent of environmental exposure. (Hennekens, C H et al., American Heart Journal (2005). Antipsychotics may significantly increase the cardiometabolic risk schizophrenic patients incur, such as effects on weight and BMI, glucose and insulin levels, hypertension, and total cholesterol and triglycerides. (Newcomer, J W, Journal of Clin. Psychiatry (2007). Such effects can compromise the effectiveness, such as, for example, the efficacy, safety, and/or tolerability, of antipsychotics. Thus, there is a public health need for novel antipsychotic therapies exhibiting a benign metabolic profile.

It is also known that antipsychotic therapies may lead to adverse effects such as, for example, cognitive impairment, extrapyramidal side effects, and sedation. Accordingly, there is an ongoing need to improve the quality of life and well-being of patients treated for schizophrenia and other CNS disorders in addition to reversing impaired cognitive functioning of patients.

It is also known that response to antipsychotic therapy varies between patients. For example, it is known that there may be a delayed effect in the treatment of patients with a CNS disorder, such as schizophrenia, between the start of antipsychotic therapies and the improvement in psychiatric symptoms. (See e.g., Kinon et al., Neuropsychopharmacology, 35:581-590 (2010)). As a result of the delayed onset of therapy, a waiting period of 4 to 8 weeks may be recommended before switching patients to other antipsychotic therapies if they are not responding to the initial therapy. (Kinon et al., Schizophrenia Research, 102:230-240 (2008)). However, it has also been shown that response to antipsychotic therapies can rapidly occur in the first 1 to 2 weeks. (Stauffer et al., Psychiatry Research, 187: 42-48 (2011)). Such variations in response can have significant implications in subsequent reduction of psychiatric symptoms.

Thus, there is a need in the art for determining the relationship between initial response to antipsychotic therapy, for example, treatment with lurasidone, and subsequent response to the therapy in the treatment of CNS disorders such as, for example, mixed depression, schizophrenia, and bipolar disorder, and also for adjusting dose(s) and dosage intervals in certain patients in view of that determination in order to improve the safety and/or efficacy of treatment.

Disclosed herein are methods of treating, preventing and/or managing at least one CNS disorder comprising administering to a patient a therapeutically or prophylactically effective amount of at least one compound chosen from lurasidone and pharmaceutically acceptable salts, solvates, clathrates, and stereoisomers thereof, such as chosen from lurasidone and a pharmaceutically acceptable salt thereof.

Also disclosed herein are dosing regimens for treatment with at least one compound chosen from lurasidone and pharmaceutically acceptable salts, solvates, clathrates, and stereoisomers thereof.

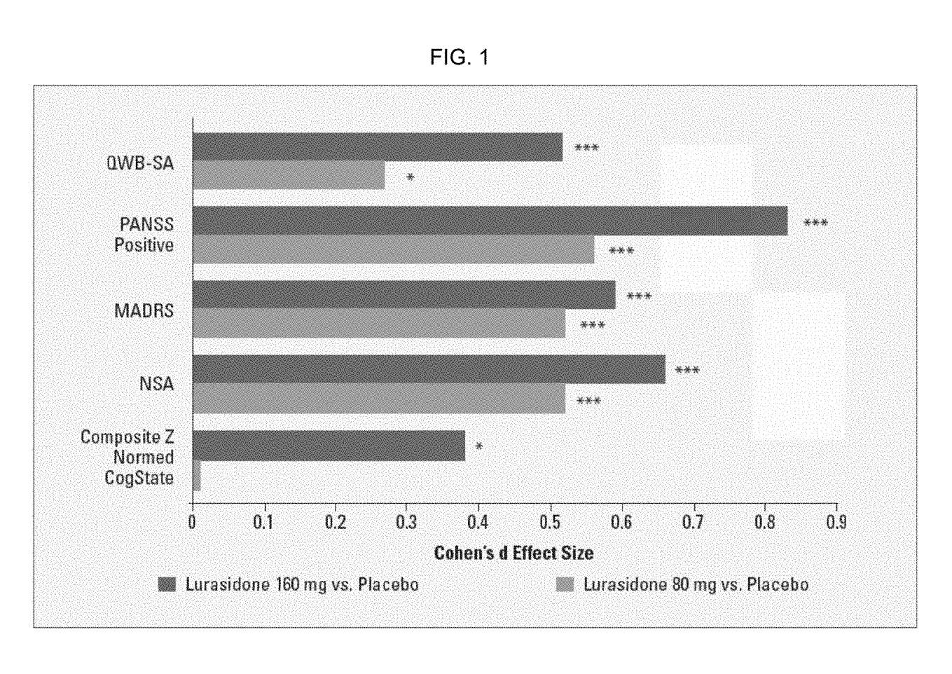

FIG. 1 illustrates improvement in Quality of Well-Being Self Administered questionnaire (QWB-SA) score in patients with schizophrenia after 6 weeks of treatment with lurasidone (80 mg and 160 mg) and placebo.

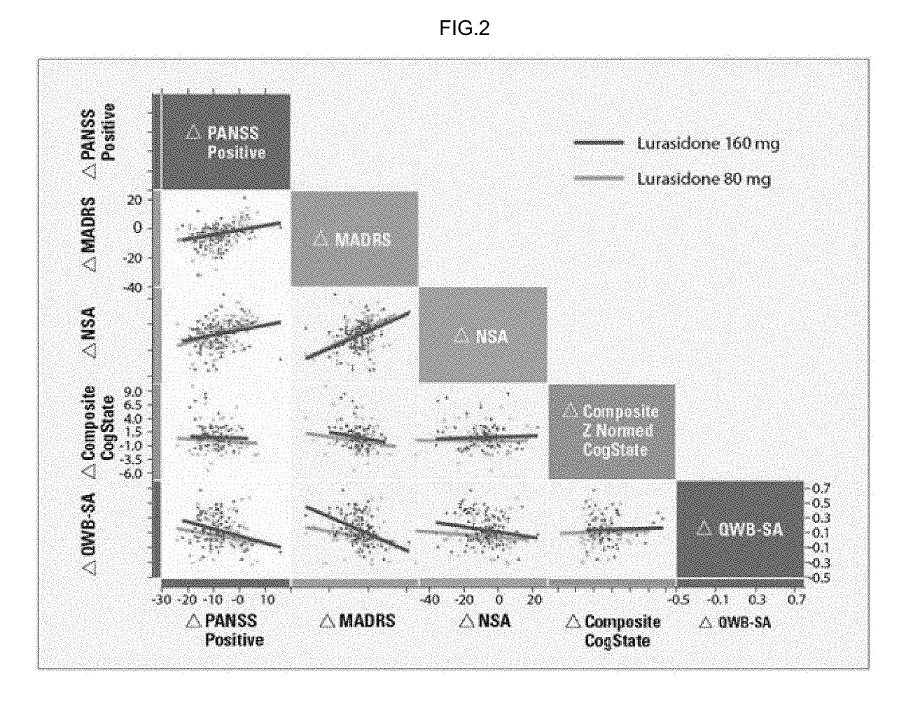

FIG. 2 illustrates the correlation between QWB-SA scores and other outcome measurements in patients with schizophrenia after 6 weeks of treatment with lurasidone (80 mg and 160 mg) and placebo.

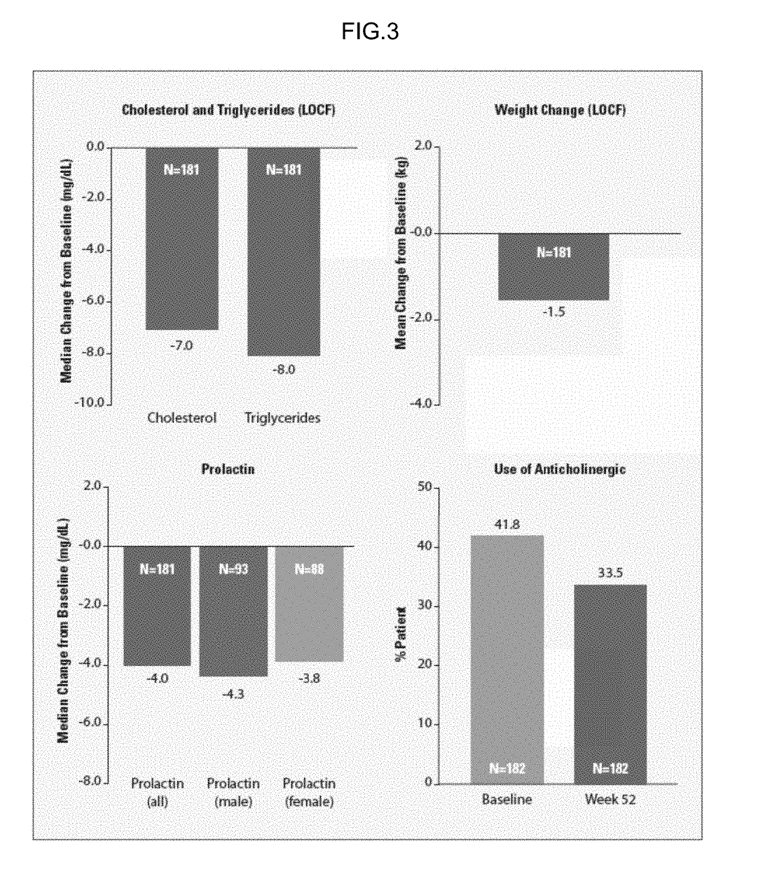

FIG. 3 illustrates the change in weight, lipids and tolerability parameters after 1 year of treatment with lurasidone (40-120 mg).

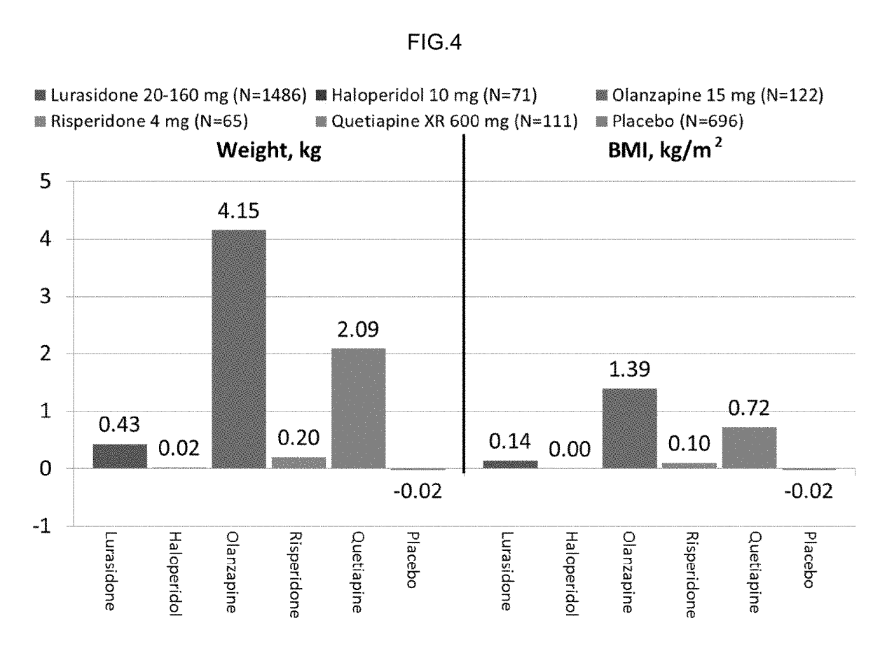

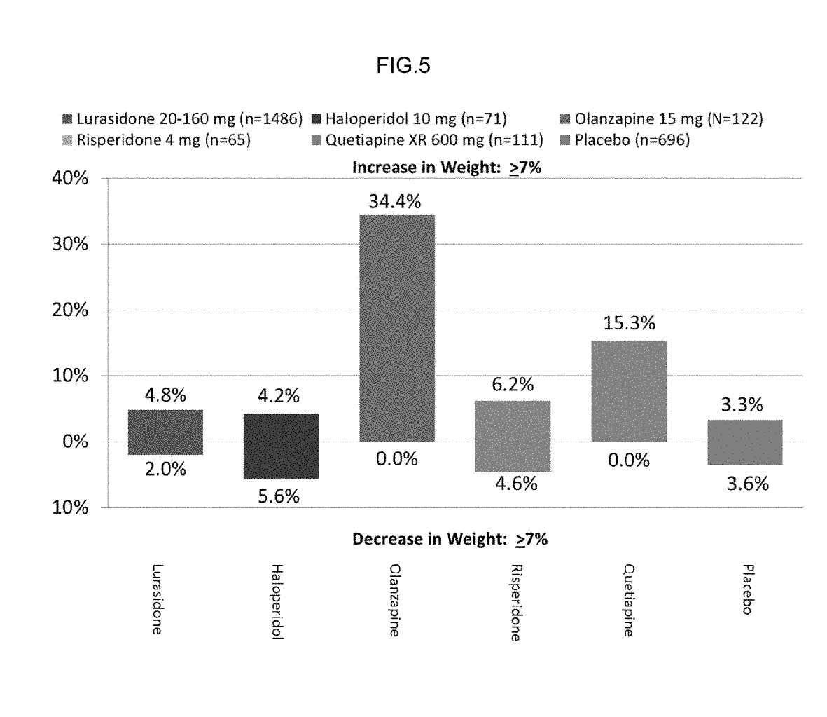

FIG. 4 illustrates the mean change in weight and BMI at 6 weeks after the initiation of treatment by lurasidone and other currently available antipsychotics.

FIG. 5 illustrates the proportion of patients with clinically significant weight gain or weight loss at 6 weeks after the initiation of treatment by lurasidone and other currently available antipsychotics.

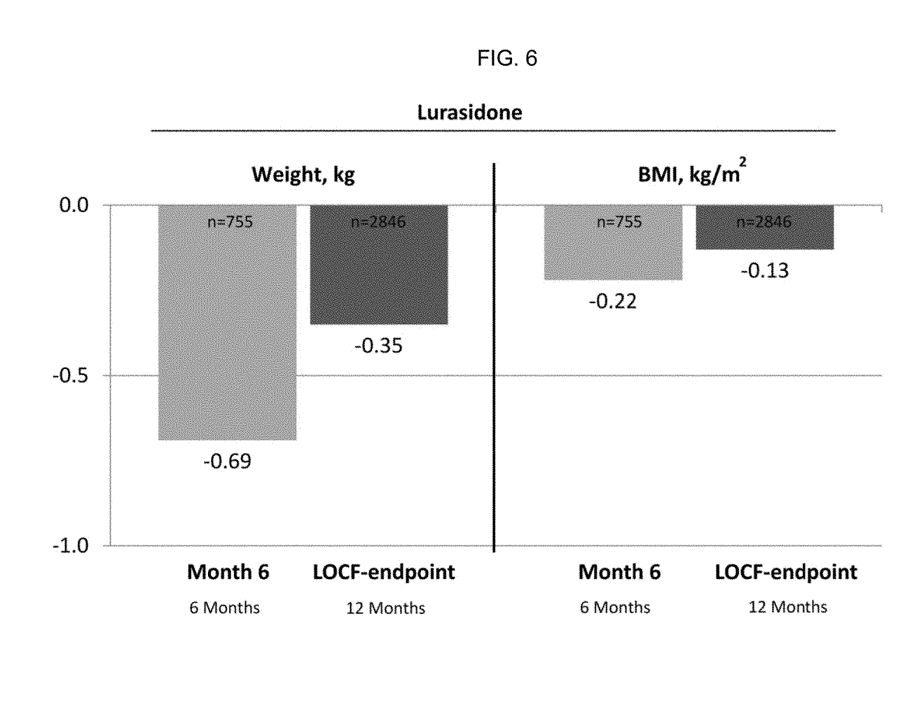

FIG. 6 illustrates the mean change in weight and BMI at 6 months and 12 months after the initiation of treatment by lurasidone.

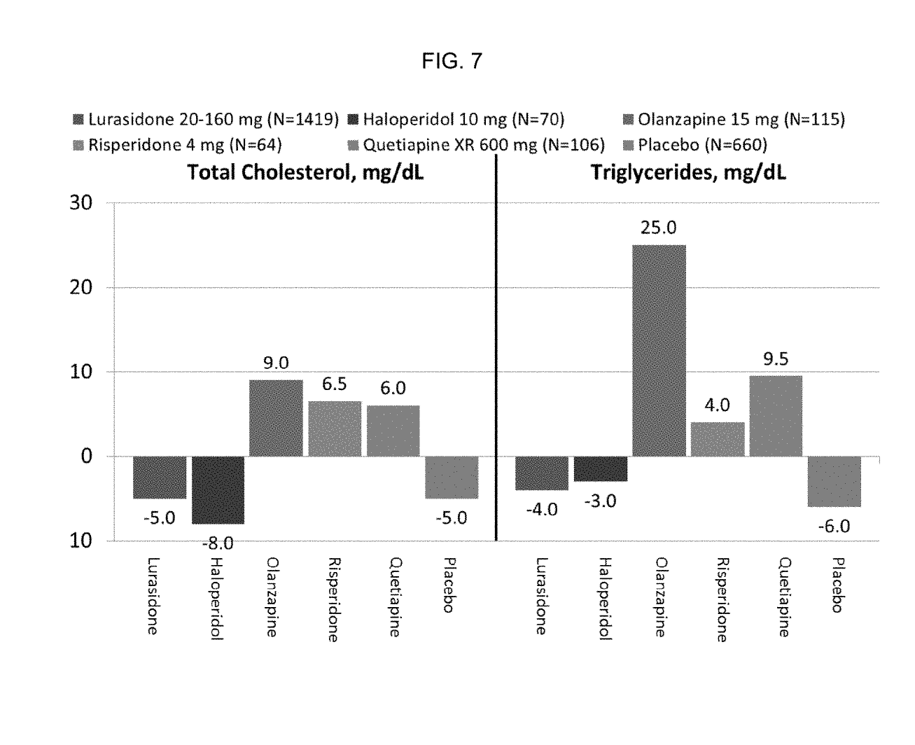

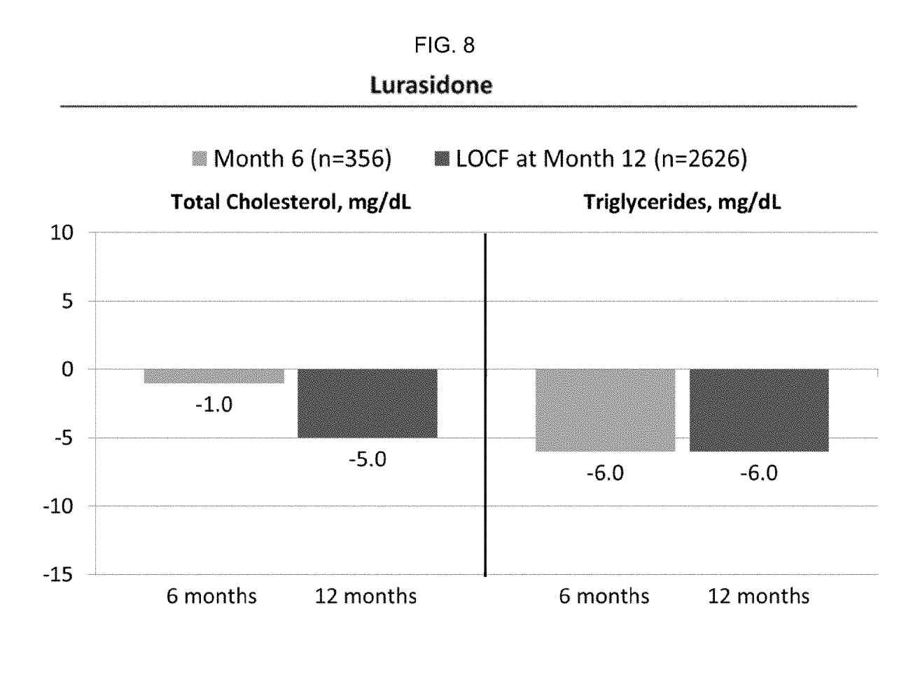

FIG. 7 illustrates the median change in total cholesterol and triglycerides at 6 weeks after the initiation of treatment by lurasidone and other currently available antipsychotics.

FIG. 8 illustrates the median change in total cholesterol and triglycerides at 6 months and 12 months after the initiation of treatment by lurasidone.

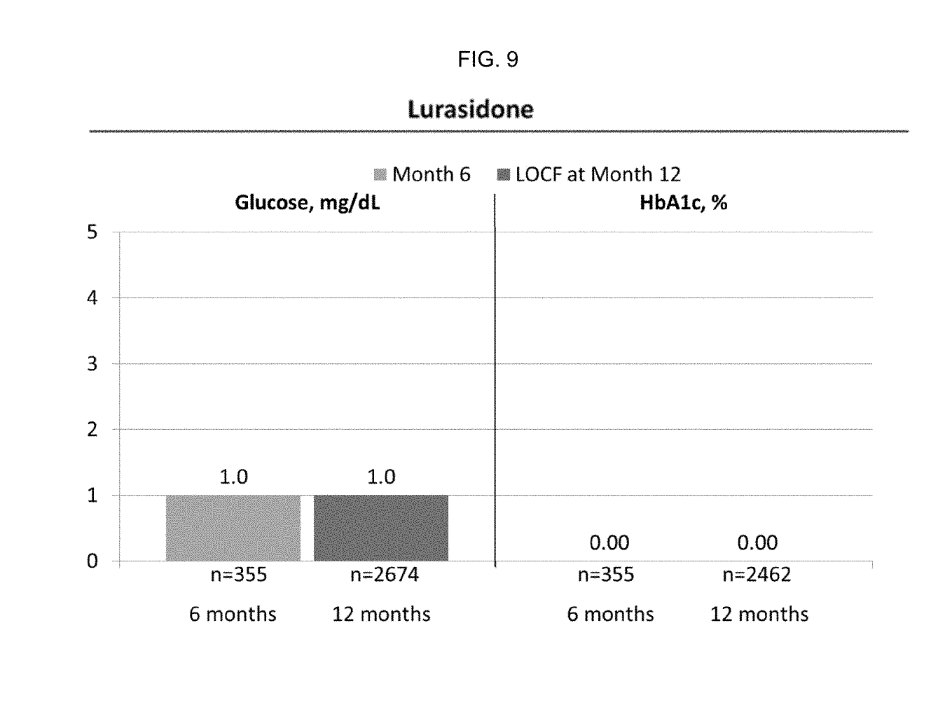

FIG. 9 illustrates the median change in glucose and HbA1c at 6 months and 12 months after the initiation of treatment by lurasidone.

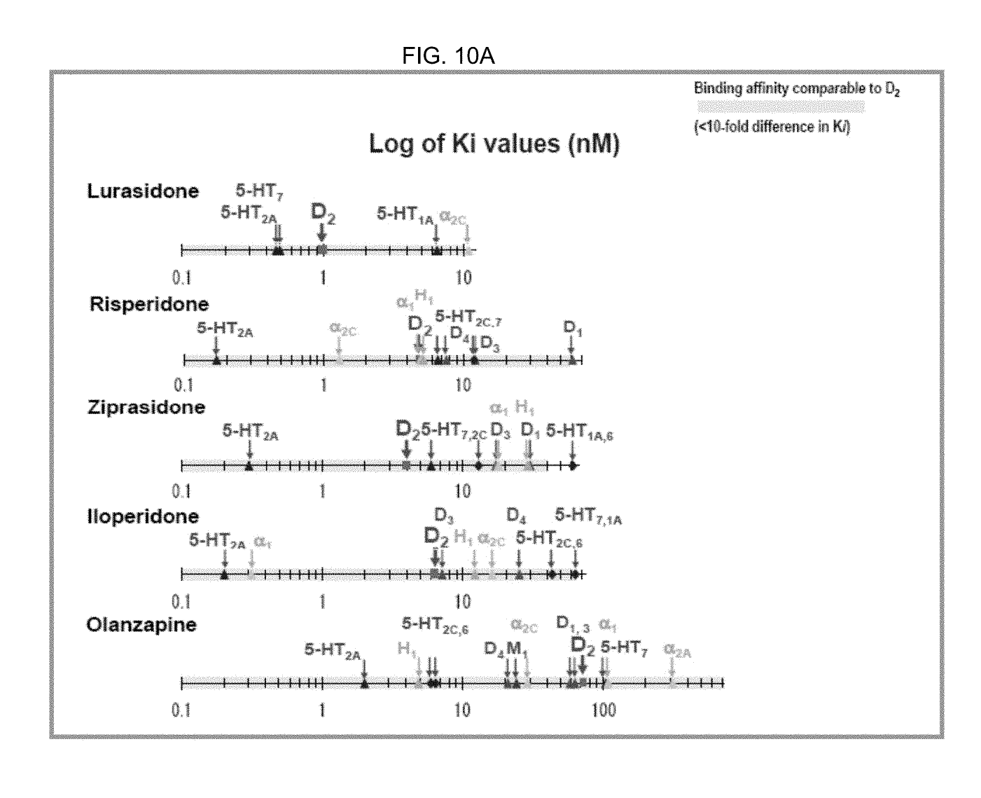

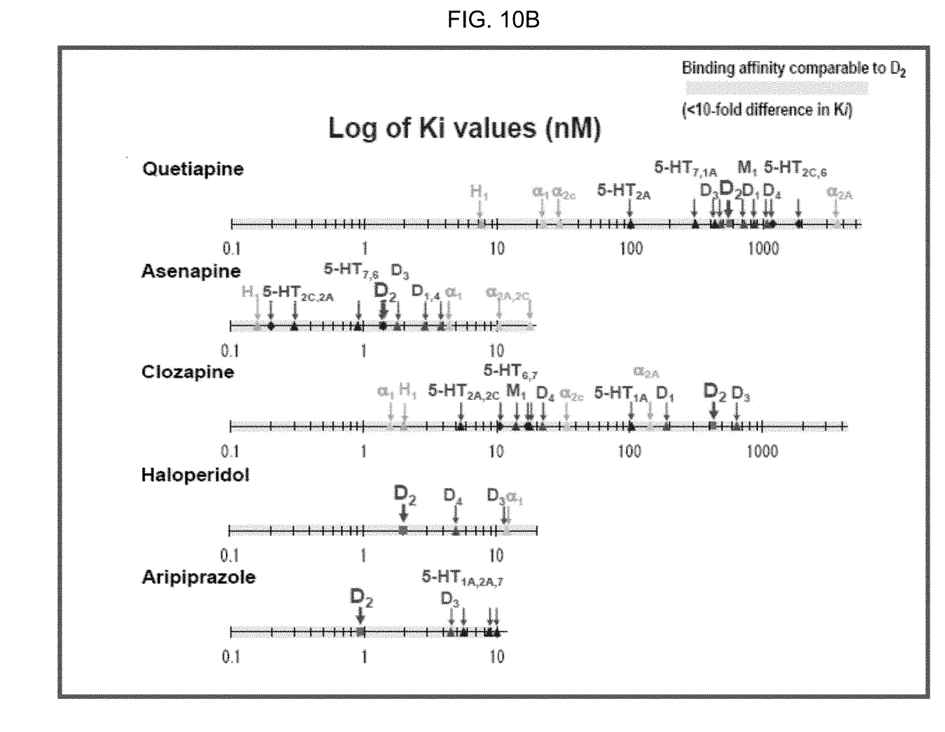

FIG. 10 illustrates the comparative receptor binding affinities of lurasidone and other currently available antipsychotics.

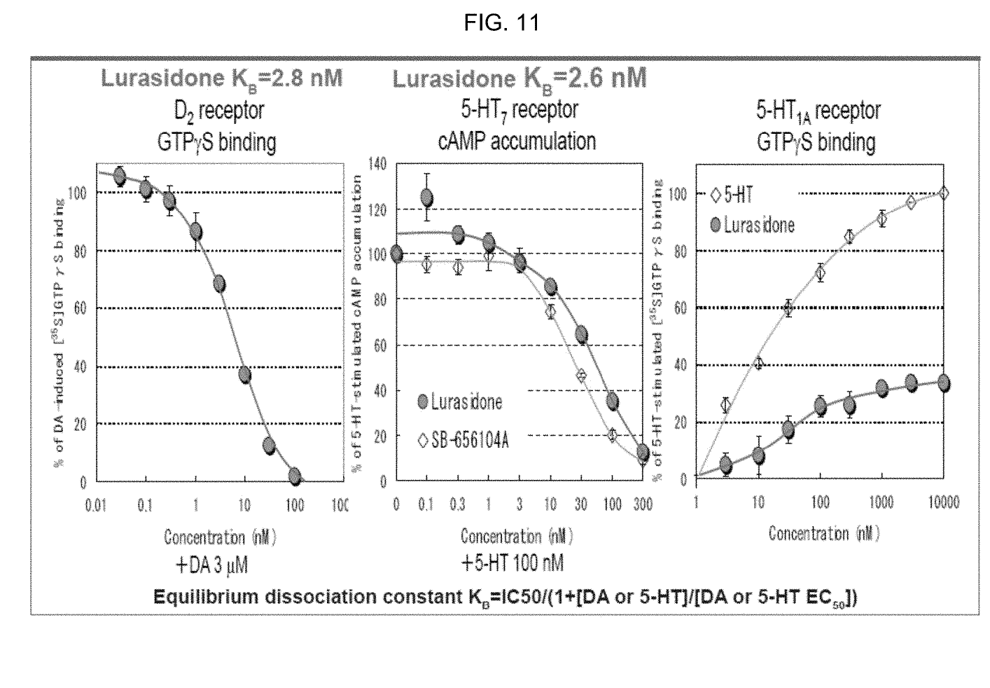

FIG. 11 illustrates the effects of lurasidone on receptor function (D₂, 5-HT₇, 5-HT_{1A}).

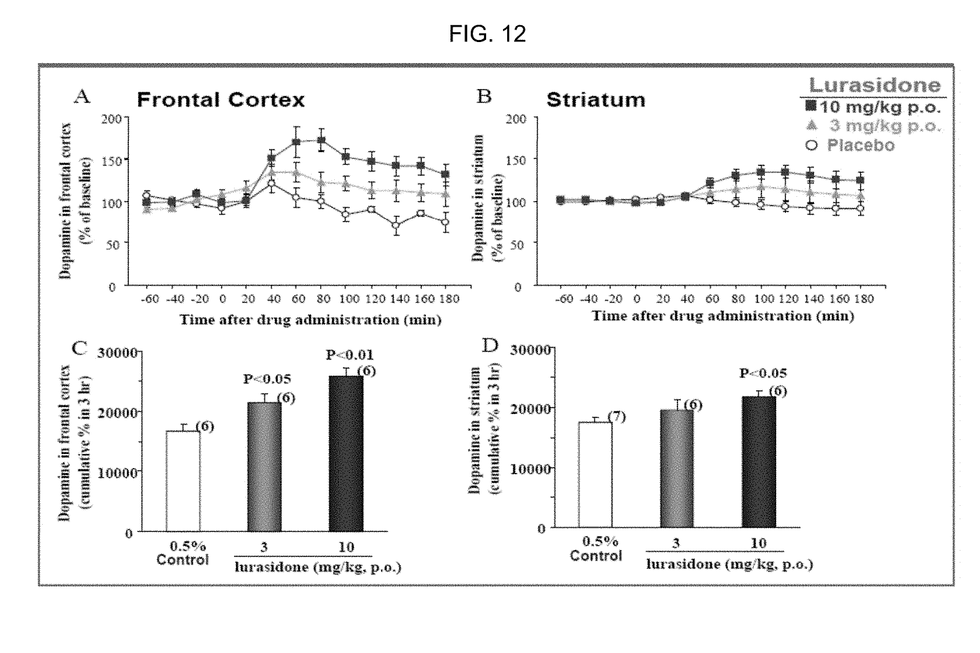

FIG. 12 illustrates the effects of lurasidone on D₂ receptor function in the frontal cortex and striatum.

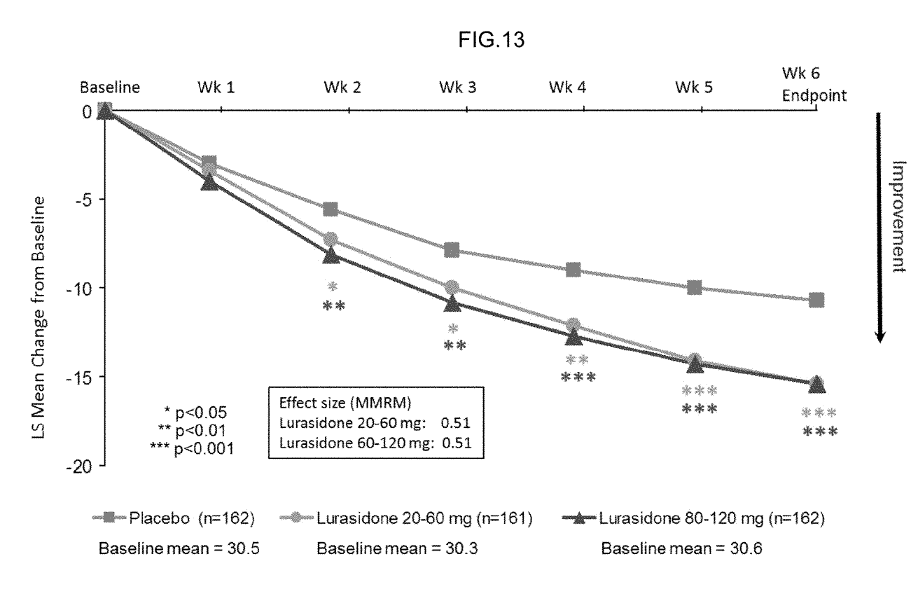

FIG. 13 illustrates the change from baseline in Montgomery-Asberg Depression Rating Scale (MADRS) total score (analyzed using mixed model for repeated measures (MMRM)) in patients treated with 20 mg/day to 60 mg/day of lurasidone, 80 mg/day to 120 mg/day of lurasidone, or placebo.

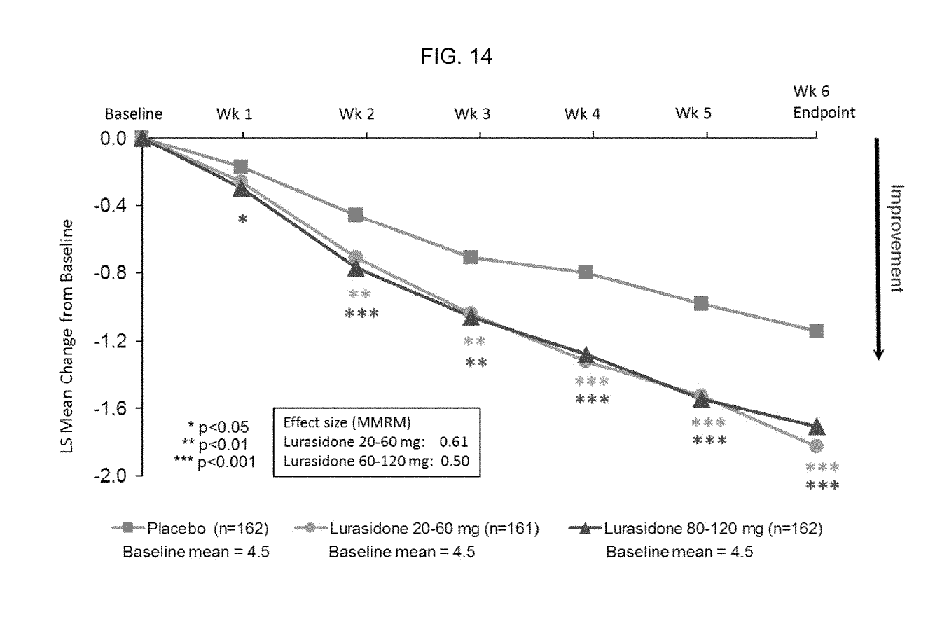

FIG. 14 illustrates the change from baseline in CGI-BP-S depression score (MMRM) in in patients treated with 20 mg/day to 60 mg/day of lurasidone, 80 mag/day to 120 mg/day of lurasidone, or placebo.

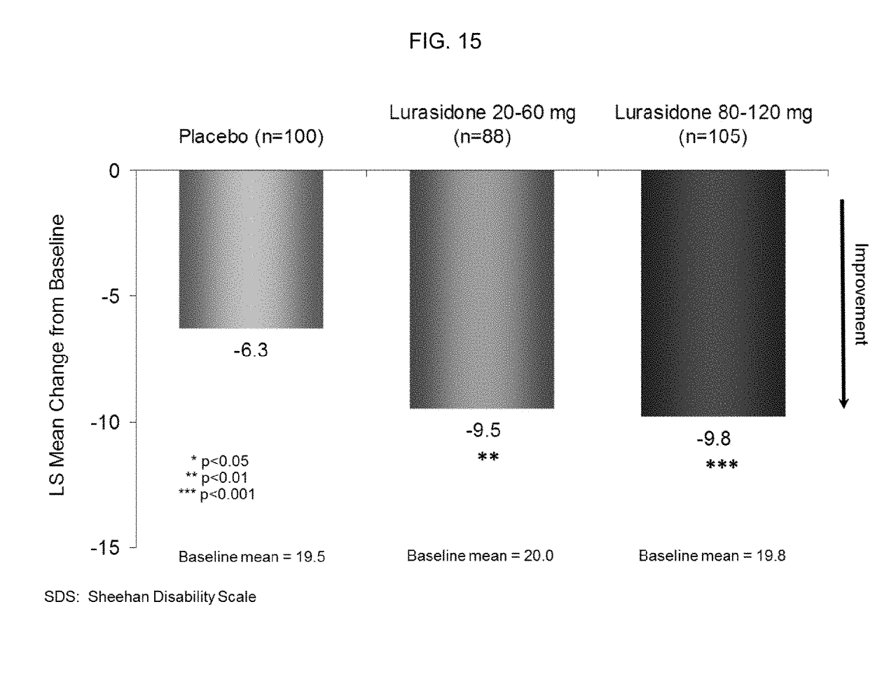

FIG. 15 illustrates the change from baseline in Sheehan Disability Scale (SDS) total score (LOCF) in patients treated with 20 mg/day to 60 mg/day of lurasidone, 80 mg/day to 120 mg/day of lurasidone, or placebo.

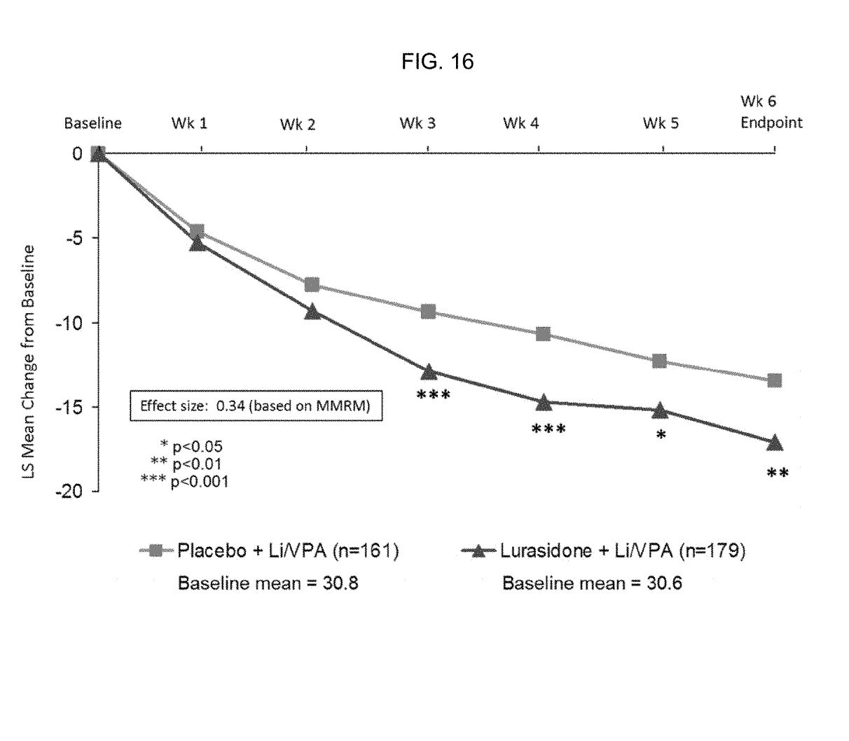

FIG. 16 illustrates the change from baseline in MADRS total score (MMRM) in patients treated with 20 mg/day to 120 mg/day of lurasidone in combination with lithium or divalproex, or placebo in combination with lithium or divalproex.

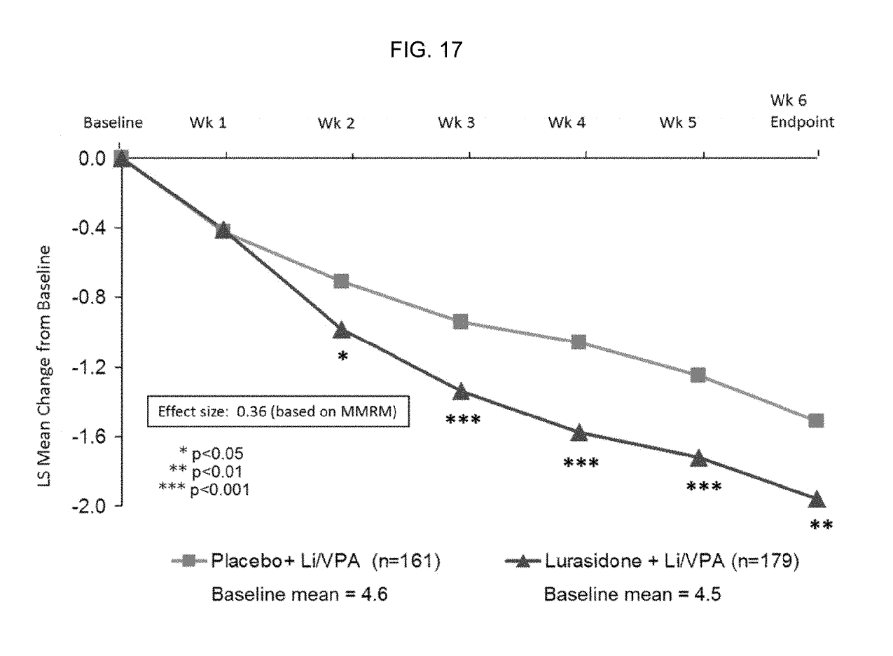

FIG. 17 illustrates the change from baseline in CGI-BP-S depression score (MMRM) in patients treated with 20 mg/day to 120 mg/day of lurasidone in combination with lithium or divalproex, or placebo in combination with lithium or divalproex.

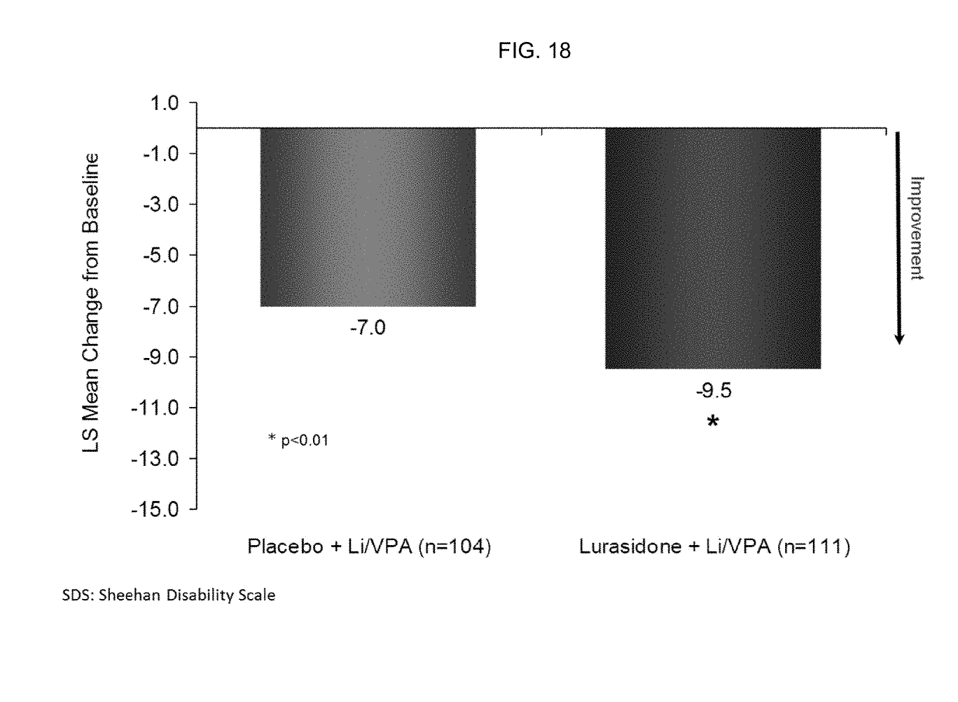

FIG. 18 illustrates the change in SDS total score (LOCF) in patients treated with 20 mg/day to 120 mg/day of lurasidone in combination with lithium or divalproex, or placebo in combination with lithium or divalproex.

In certain embodiments, at least one compound chosen from lurasidone and pharmaceutically acceptable salts, solvates, clathrates, and stereoisomers thereof may include lurasidone in free base form, one or more pharmaceutically acceptable salts of lurasidone, one or more pharmaceutically acceptable solvates of lurasidone, one or more pharmaceutically acceptable clathrates of lurasidone, and/or one or more pharmaceutically acceptable stereoisomers of lurasidone. In certain embodiments, at least one compound chosen from lurasidone and pharmaceutically acceptable salts, solvates, clathrates, and stereoisomers thereof may also include pharmaceutically acceptable salts of any of the foregoing (e.g., pharmaceutically acceptable solvates of lurasidone), pharmaceutically acceptable solvates of any of the foregoing (e.g., pharmaceutically acceptable salts of lurasidone), pharmaceutically acceptable clathrates of any of the foregoing, and/or pharmaceutically acceptable stereoisomers of any of the foregoing.

In certain embodiments, the “at least one compound” refers to lurasidone which may be in the form, for example, of its free base, a pharmaceutically acceptable salt, a solvate, a clathrate, a stereoisomer and/or a mixture of any of the foregoing forms. In certain embodiments, the at least one compound is chosen from lurasidone and a pharmaceutically acceptable salt thereof. In certain embodiments, the at least one compound is a pharmaceutically acceptable salt of lurasidone. In certain embodiments, the at least one compound is lurasidone hydrochloride.

As used herein, unless otherwise specified, the term a “pharmaceutically acceptable salt” refers to a salt prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucuronic, galacturonic, glycidic, hydrobromic, hydrochloric, isethionic, lactic, maleic, malic, mandelic, methanesulfonic, mucic, nitric pamoic, pantothenic, phenylacetic, propionic, phosphoric, salicylic, stearin, succinic, sulfanilic, sulfuric, tartaric, p-toluenesulfonic acid and the like. In one embodiment, suitable acids are chosen from hydrochloric acid, hydrobromic acid, phosphoric acid, and sulfuric acid.

As used herein, unless otherwise specified, the term “solvate” means a compound that further includes a stoichiometric or no amount of solvent bound by noncovalent intermolecular forces. Where the solvent is water, the solvate is called a hydrate.

As used herein, unless otherwise specified, the term “stereoisomer” encompasses all enantiomerically and/or stereomerically pure and enantiomerically and/or stereomerically enriched compounds disclosed herein.

As used herein, unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, greater than about 98% by weight of one stereoisomer of the compound and less than about 2% by weight of the other stereoisomers of the compound or greater than about 99% by weight of one stereoisomer of the compound and less than about 1% by weight of the other stereoisomers of the compound.

As used herein, unless otherwise indicated, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, unless otherwise specified, the terms “treat,” “treating” and “treatment” refer to an action in a patient suffering from at least one CNS disorder disclosed herein which inhibits, arrests, and/or relieves (retards or slows) the severity of the CNS disorder and/or the symptoms associated therewith.

As used herein, unless otherwise specified, the terms “prevent,” “preventing” and “prevention” refer to treatment with or administration of at least one compound disclosed herein prior to the onset of at least one symptom, for example, in patients at risk of at least one CNS disorder disclosed herein. The term “prevention” includes the inhibition and/or preclusion of at least one symptom of a particular disorder. Patients with familial history of a disease in particular are candidates for preventive regimens in some embodiments. In some embodiments, patients with a history of recurring symptoms are candidates for preventive regimens. In this regard, the term “prevention” may be interchangeable with the term “prophylactic treatment.”

As used herein, unless otherwise specified, the terms “manage,” “managing” and “management” refer to preventing or slowing the progression, spread and/or worsening of at least one disorder or of at least one symptom thereof. In some embodiments, the effect(s) of a prophylactic or therapeutic agent may not result in a cure of the at least one disorder.

As used herein, unless otherwise specified, the term “therapeutically effective amount” is an amount sufficient to provide at least one therapeutic benefit in the treatment or management of at least one CNS disorder disclosed herein or sufficient to delay or minimize at least one symptom associated with at least one CNS disorder disclosed herein. A therapeutically effective amount of at least one compound means an amount of the at least one compound which, alone or in combination with at least one other therapeutic agent, can provide at least one therapeutic benefit in the treatment or management of at least one CNS disorder disclosed herein. The term “therapeutically effective amount” therefore encompasses an amount that improves overall therapy, an amount that reduces at least one symptom of at least one CNS disorder disclosed herein, an amount that reduces at least one cause of at least one CNS disorder disclosed herein, and/or an amount that enhances the therapeutic efficacy of at least one other therapeutic agent.

As used herein, unless otherwise specified, a “prophylactically effective amount” of at least one compound is an amount sufficient to inhibit/reduce at least one symptom of at least one CNS disorder disclosed herein or an amount sufficient to prevent recurrence of at least one CNS disorder disclosed herein. A prophylactically effective amount of at least one compound means an amount of at least one compound that, alone or in combination with at least one other agent, can provide at least one prophylactic benefit in the inhibition/reduction of at least one symptom of at least one CNS disorder disclosed herein and/or in the recurrence of at least one CNS disorder disclosed herein. The term “prophylactically effective amount” encompasses an amount that improves overall prophylaxis and/or an amount that enhances the prophylactic efficacy of at least one other prophylactic agent.

As used herein, unless otherwise specified, the term “about,” when used in connection with a specific value, means that acceptable deviations from that value are also encompassed. In certain embodiments, the term “about” means that a value higher or lower than the given value by 1%, 3%, 5% 10%, 15%, 20%, 25%, 30%, 35% or 40% is encompassed.

The term “predict” generally means to determine or tell in advance. When used to “predict” the effectiveness of the treatment of at least one CNS disorder disclosed herein, for example, the term “predict” can mean that the likelihood of the outcome of the treatment can be determined at the outset, before the treatment has begun, or before the treatment period has progressed substantially.

The term “likelihood” generally refers to an increase in the probability of an event. The term “likelihood” when used in reference to the effectiveness of a patient response generally contemplates an increased probability that the symptoms of a CNS disorder disclosed herein will be lessened or decreased.

As used herein, the terms that refer to at least one CNS disorders disclosed herein elsewhere are used herein in a manner consistent with their accepted meanings in the art. See, e.g., Diagnostic and Statistical Manual of Mental Disorders, 4th Ed., American Psychiatric Association (1997) (DSM IV™).

As used herein, unless otherwise indicated, the term “modulator,” when used in connection with certain biochemicals or receptors, means an agent that can increase or decrease the level of the biochemicals or the activity of the receptors.

As used herein, unless otherwise indicated, the term “increase,” when referring to level or activity, means that the level or activity can be increased, for example, by about 5%, 10%, 20%, 30%, 40%, 50%, 60%, 70%, 90%, 100%, 200%, 300%, 500%, 1,000%, 5,000% or more of the comparative control level.

As used herein, unless otherwise indicated, the term “decrease,” when referring to level or activity, means that the level or activity can be decreased, for example, by about 99%, 95%, 90%, 80%, 70%, 60%, 50%, 40%, 30%, 20%, 10%, 1% or less of the comparative control level.

The terms “determining”, “measuring”, “evaluating”, “assessing” and “assaying” as used herein, unless otherwise indicated, generally refer to any form of measurement, and include determining if an element is present or not. These terms include both quantitative and/or qualitative determinations. Assessing may be relative or absolute.

The term “sample” as used herein, unless otherwise indicated, relates to a material or mixture of materials, typically, although not necessarily, in fluid form, containing one or more components of interest.

“Biological sample” as used herein, unless otherwise indicated, refers to a sample obtained from a biological subject, including sample of biological tissue or fluid origin, obtained, reached, or collected in vivo or in situ. Such samples can be, but are not limited to, organs, tissues, fractions, sera and cells isolated from a mammal (e.g., human). Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, PBMCs, tissue biopsies, and the like.

After metabolomic profiling of samples obtained from patients who had been treated with at least one compound, the at least one compound being chosen from, for example, lurasidone, a pharmaceutically acceptable salt, solvate, clathrate and stereoisomer thereof, minimal or no metabolic side effects or weight gain were observed. Embodiments disclosed herein are based, in part, on the unexpected discovery that the administration of the at least one may result in improved quality of life and/or may reverse impairment in learning and memory associated with schizophrenia.

Accordingly, in sonic embodiments, disclosed herein are methods of treating, preventing and/or managing at least one CNS disorder comprising administering to a patient a therapeutically or prophylactically effective amount of at least one compound chosen from lurasidone and pharmaceutically acceptable salts, solvates, clathrates and stereoisomers thereof.

Examples of at least one CNS disorder include, but are not limited to, adjunctive therapy in bipolar disorder, adjunctive therapy in depression, adolescent bipolar disorder, adolescent depression, alcohol dependence, Alzheimer's disease, anorexia nervosa, attention deficit disorder, an anxiety disorder including generalized anxiety disorder and social anxiety disorder, bipolar disorder, bipolar maintenance, borderline personality disorder, childhood schizophrenia, circadian rhythm sleep disorder, cognitive function impairment, cognitive impairment associated with schizophrenia, cognitive remediation therapy, conduct disorder, delirium, depression maintenance, first episode psychosis, Fragile-X Syndrome, mixed depression, monotherapy in bipolar disorder, monotherapy in depression, neuropathic pain, obsessive compulsive disorder, panic disorder, pathological gambling, post-traumatic stress disorder, Prodromal Risk Syndrome, psychosocial function impairment, psychotic depression, quality of life in schizophrenia, bipolar disorder and depression, schizophrenia, negative symptoms associated with schizophrenia, schizophrenia maintenance, schizoaffective disorder, sleep disorder, social functioning impairment, substance abuse, treatment resistant depression, treatment resistant schizophrenia, and Tourette's Disorder. In an embodiment, the at least one CNS disorder is Alzheimer's disease, bipolar disorder, mixed depression, schizophrenia, or negative symptoms associated with schizophrenia.

In an embodiment, the at least one CNS disorder is bipolar disorder. As used herein, bipolar disorder, unless otherwise indicated, is at least one bipolar disorder chosen from disorders such as bipolar I disorder, bipolar II disorder, and cyclothymic disorder.

In an embodiment, the at least one CNS disorder is mixed depression. In one embodiment, mixed depression is depression and at least one symptom chosen from manic symptoms and hypomanic symptoms. In another embodiment, mixed depression is a major depressive disorder with mixed features, such as a major depressive episode with mixed features, such as with a limited number of manic symptoms.

In an embodiment, a major depressive episode is diagnosed according to the proposed Diagnostic and Statistical Manual of Mental Disorders (DSM-5). See Proposed revision of DSM-5 by American Psychiatric Association, http://www.dsm5.org/. Thus in some embodiments, a major depressive episode is diagnosed based on the presence of at least five of the following symptoms over a single two-week period that represent a change from previous functioning and that are not clearly due to a medical condition:
1. depressed for most of each day, nearly every day (which may be indicated by either subjective report, such as feeling sad or empty, or observation made by others, such as appears tearful) or, in children and adolescents, may also present as irritability;
2. markedly diminished interest or pleasure in all, or almost all, activities most of the day, nearly every day (as indicated by either subjective account or observation made by others);
3. significant weight loss when not dieting or weight gain (e.g., a change of more than 5% of body weight in a month), decrease or increase in appetite nearly every day, or, in children, failure to make expected weight gain;
4. insomnia or hypersomnia nearly every day;
5. psychomotor agitation or retardation nearly every day (observable by others, not merely subjective feelings of restlessness or being slowed down);
6. fatigue or loss of energy nearly every day;
7. feelings of worthlessness or excessive or inappropriate guilt (which may be delusional) nearly every day (not merely self-reproach or guilt about being sick);
8. diminished ability to think or concentrate, or indecisiveness, nearly every day (either by subjective account or as observed by others);
9. recurrent thoughts of death (not just fear of dying), recurrent suicidal ideation without a specific plan, a suicide attempt, or a specific plan for committing suicide.

In at least one embodiment, at least one of the symptoms is criteria (1) above (depressed mood (or possibly irritability in children and adolescents)) or criteria (2) above (markedly diminished interest or pleasure). See “Proposed Criteria for Major Depressive Episode” (http://www.dsm5.org/ProposedRevisions/Pages/proposedrevision.aspx?rid=427#, Updated Oct. 12, 2010)

In at least one embodiment, if five or more symptoms indicating a major depressive episode are present, mixed depression is diagnosed if the full criteria are met for a major depressive episode (see above) and at least three of the following mixed features specifiers or symptoms are present nearly every day during the major depressive episode:
Elevated, expansive mood
Inflated self-esteem or grandiosity
More talkative than usual or pressure to keep talking
Flight of ideas or subjective experience that thoughts are racing
Increase in energy or goal directed activity (either socially, at work or school, or sexually)
Increased or excessive involvement in activities that have a high potential for painful consequences (e.g., engaging in unrestrained buying sprees, sexual indiscretions, or foolish business investments).
Decreased need for sleep (feeling rested despite sleeping less than usual (to be contrasted from insomnia)

### See “Proposed Criteria for Mixed Features Specifier”

(http://www.dsm5.org/ProposedRevisions/Pages/proposed [symbol] revision.aspx?rid=483).

In at least one embodiment, mixed depression is diagnosed when the subject is (1) currently experiencing a major depressive episode defined according to the diagnostic criteria set forth in DSM-IV-TR (Diagnostic and Statistical Manual of Mental Disorders, 4th Ed., Text Revision; American Psychiatric Association, “Diagnostic and Statistical Manual of Mental Disorders DSM-IV-TR Fourth Edition (Text Revision)”, 2000) for at least two weeks in duration and (2) experienced two or three of the mixed features specifiers listed on most days over at least the last two weeks. In at least one embodiment, mixed depression also includes the criteria that the patient is between the ages of 18 and 75 and/or has a MADRS (Montgomery-Asberg Depression Rating Scale score) total score of greater than or equal to 26 at screening and baseline visits. The MADRS is a clinician-rated assessment of the subject's level of depression. The measure contains 10 items that measure apparent and reported sadness, inner tension, reduced sleep and appetite, difficulty concentrating, lassitude, inability to feel, and pessimistic and suicidal thoughts. Each item is scored in a range of 0 to 6 points, with higher scores indicating increased depressive symptoms. (S A Montgomery and M Asberg, “A new depression scale designed to be sensitive to change,” The British Journal of Psychiatry (1979) 134: 382-389.)

In another embodiment, mixed depression does not include patients with at least one of the following: (1) patients with an Axis I or Axis II diagnosis (DSM-IV) other than major depressive disorder that has been the primary focus of treatment within the three months prior to screening, (2) patients that answer “yes” to “suicidal ideation” on items 4 or 5 of the C-SSRS (Columbia Suicide Severity Rating Scale);

http://www.cssrs.columbia.edu; FDA, “Guidance for Industry, Suicidality: Prospective Assessment of Occurrence in Clinical Trials, DRAFT GUIDANCE”, September 2010, http://www.fda.gov/downloads/Drugs/GuidanceComplianceRegulatoryinformation/Guidances/UCM225130.pdf), (3) patients that have a lifetime history of any bipolar I manic or mixed manic episode, and (4) patients that have any abnormal laboratory parameter at screening that indicates a clinically significant medical condition as determined by the investigator.

In an embodiment, patients who meet entry criteria of mixed depression described above, are randomly assigned (1:1) to either lurasidone (approximately 95 patients) or placebo (approximately 95 patients), in a double-blind fashion. Patients randomized to the lurasidone arm are treated with lurasidone hydrochloride 20 mg/day from Day 1 to Day 7. Flexible dosing of study drug (20 mg, 40 mg, or 50 mg/day) is permitted after 7 days (beginning on Day 8) to optimize efficacy and tolerability. Dose adjustments may occur at weekly intervals and in increments/decrements of one dose level. Dose reductions for tolerability or safety purposes are permitted to occur more frequently than at weekly intervals and more than one dose level at a time (maximum of two dose levels at a time), beginning at Day 8. The primary efficacy endpoint is the mean change from baseline in MADRS total score after 6 weeks of treatment (Day 43). The key secondary endpoint is the mean change from baseline in the Clinical Global Impression-Severity of Illness (CGI-S) score after 6 weeks of treatment (Day 43).

In an embodiment, the at least one CNS disorder is schizophrenia. In another embodiment, the at least one CNS disorder is negative symptoms associated with schizophrenia. Examples of negative symptoms associated with schizophrenia include, but are not limited to, extrapyramidal symptoms, tardive dyskinesia, sedation, and metabolic side effects such as, for example, weight gain, hyperglycemia, hyperlipidemia, hypotension, cardiac disease, and diabetes.

In an embodiment, the at least one CNS disorder is chosen from learning and memory impairment and cognitive impairment associated with schizophrenia. Examples of learning and memory impairment include, but are not limited to, decline in cognitive functions or cognitive domains, e.g., working memory, attention and vigilance, verbal learning and memory, visual learning and memory, reasoning and problem solving, e.g., executive function and/or speed of processing.

Thus, disclosed herein are methods of treating, preventing and/or managing at least one CNS disorder comprising administering to a patient who received a prior therapy a therapeutically or prophylactically effective amount of at least one compound chosen from lurasidone and pharmaceutically acceptable salts, solvates, clathrates and stereoisomers thereof. Examples of CNS disorders are disclosed above.

Also disclosed herein are methods of improving cognitive impairment, for example, in patients with at least one CNS disorder and/or in patients undergoing treatment for at least one CNS disorder. In an embodiment, improvement in cognitive impairment includes at least one improvement chosen from verbal learning improvements, visuospatial learning improvements, memory attention improvements, improvements in speed of processing, and improvements in motor skills.

Also disclosed herein are methods of preventing development of cognitive impairment in patients who are at risk of developing at least one CNS disorder, such as schizophrenia and/or psychosis.

Also disclosed herein are methods of reducing side effects associated with at least one CNS disorder and/or treatment of at least one CNS disorder such as, for example, reducing daytime sleepiness and/or metabolic side effects.

In at least one embodiment, methods for reducing metabolic side effects include, but are not limited to reducing weight gain and/or maintaining benign levels of cholesterol, low-density and high-density lipoproteins, triglycerides, insulin, glycosylated hemoglobin, and/or glucose. In at least one embodiment, methods of lowering levels of cholesterol and/or triglycerides are disclosed.

Also disclosed herein are methods of treating, preventing and/or managing at least one CNS disorder without an increase of daytime sleepiness, and in at least one embodiment, the at least one CNS disorder is schizophrenia. In other embodiments, the at least one CNS disorder is bipolar depression or mixed depression. Yet, in other embodiments, the methods of treating, preventing and/or managing at least one CNS disorder occur without a substantial increase of daytime sleepiness.

The term “substantial increase of daytime sleepiness,” as used herein, unless otherwise indicated, relates to a score of greater than zero, such as greater than 0.5, on the Epsworth Sleepiness Scale (measuring the chance of dozing while sitting and reading, watching television, sitting inactive in a public place, as a passenger in a car without a break, afternoon resting, while talking with someone, sitting quietly after lunch without alcohol, and in a car, stopped for a few minutes in traffic).

Further disclosed herein are methods of treating, preventing and/or managing at least one CNS disorder with decrease of daytime sleepiness, and in at least one embodiment, the at least one CNS disorder is schizophrenia. In other embodiments, the at least one CNS disorder is bipolar depression or mixed depression.

Also disclosed herein are methods of improving at least one symptom selected from positive symptoms, negative symptoms and cognitive impairment in patients with at least one CNS disorder without substantial increase of daytime sleepiness. In at least one embodiment, the at least one CNS disorder is schizophrenia, bipolar disorder, or mixed depression.

Further disclosed herein are methods of reducing risk of relapse of at least one CNS disorder. In at least one embodiment, the at least one CNS disorder is schizophrenia, bipolar disorder, or mixed depression.

Also disclosed herein are methods of preventing relapse of at least one CNS disorder, and in at least one embodiment, the at least one CNS disorder is schizophrenia.

In some embodiments, a pharmaceutically acceptable salt, solvate, clathrate or stereoisomer of lurasidone is used.

In an embodiment, a pharmaceutically acceptable salt of lurasidone is used. In an embodiment, a hydrochloride salt of lurasidone is used.

In an embodiment, a pharmaceutically acceptable solvate of lurasidone or salt thereof is used. In an embodiment, the solvate is a hydrate.

In connection with all of the embodiments described herein, any suitable route of administration can be employed for providing a therapeutically and/or prophylactically effective dose.

The amount to be administered to a patient to treat, prevent, and/or manage the at least one CNS disorder described herein will depend upon a variety of factors including the activity of the particular compound employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health, and prior medical history of the patient being treated, and like factors well-known in the art.

In general, a suitable daily dose of the at least one compound disclosed herein will be that amount of the at least one compound that is the lowest dose effective to produce a therapeutic and/or prophylactic effect. Such an effective dose will generally depend upon the factors described above. The dosage may be formulated as a single or multiple unit dosage formulation. In an embodiment, the at least one compound is given in single or divided doses per day.

In an embodiment, the at least one compound chosen from lurasidone and pharmaceutically acceptable salts, solvates, clathrates and stereoisomers thereof may be used in an amount of about 0.1 mg to about 500 mg per day. In some embodiments, the dose may be adjusted in a conventional fashion (e.g., the same amount administered each day of the treatment, prevention or management period), in cycles (e.g., one week on, one week off), and/or in an amount that increases or decreases over the course of treatment, prevention, and/or management.

In some embodiments, the dose can be from about 1 mg to about 300 mg per day, from about 0.1 mg to about 160 mg per day, from about 1 mg to about 200 mg per day, from about 10 mg to about 120 mg per day, from about 20 mg to about 160 mg per day, from about 40 mg to about 120 mg per day, from about 10 mg to about 80 mg per day, from about 20 mg to about 80 mg per day, or from about 80 mg to about 160 mg per day. These doses can be administered in single or divided administrations.

In some embodiments, the dose can be about 10 mg per day, 20 mg per day, 30 mg per day, 40 mg per day, 50 mg per day, 60 mg per day, 70 mg per day, 80 mg per day, 90 mg per day, 100 mg per day, 110 mg per day, 120 mg per day, 130 mg per day, 140 mg per day, 150 mg per day, 160 mg per day, 170 mg per day, 180 mg per day, 190 mg per day, 200 mg per day, 240 mg per day, 280 mg per day, or 300 mg per day. These doses can be administered in single or divided administrations.

In an embodiment, the dose is about 20 mg per day. In an embodiment, the dose is about 40 mg per day. In an embodiment, the dose is about 80 mg per day. In an embodiment, the dose is about 120 mg per day. In an embodiment, the dose is about 160 mg per day. In an embodiment, the dose is about 220 mg per day. In an embodiment, the dose is about 60 mg per day.

In some embodiments, the dose can be administered as follows:
(1) Days 1 to 3: 20 mg/day, Days 4 to 6: 40 mg/day; Day 7: 60 mg/day;
(2) Days 1 to 2: 20 mg/day; Days 3 to 4: 40 mg/day; Days 5 to 6; 60 mg/day; Day 7: 80 mg/day;
(3) Days 1 to 3: 40 mg/day; Days 4 to 6: 80 mg/day; Day 7; 120 mg/day; or
(4) Day 1: 20 mg/day; Day 2: 40 mg/day; Day 3: 60 mg/day; Day 4; 80 mg/day; Day 5: 100 mg/day; Day 6: 120 mg/day.

In some embodiments, the at least one compound may be used as monotherapy. In other embodiments, the at least one compound may be used in combination with at least one additional active agent to treat, prevent, and/or manage disorders disclosed herein. In these embodiments, the at least one compound can be administered simultaneously or sequentially with the at least one additional active agent.

In at least one embodiment, the at least one additional active agent is chosen from Selective Serotonin Reuptake Inhibitors, for example, citalopram.

Pharmaceutical compositions can be used in the preparation of dosage forms useful herein. Such pharmaceutical compositions and dosage forms disclosed herein comprise at least one compound chosen from lurasidone and pharmaceutically acceptable salts, solvates, clathrates and stereoisomers thereof.

Pharmaceutical compositions and dosage forms useful herein can also comprise at least one additional active ingredient.

In an embodiment, pharmaceutical compositions and dosage forms may further comprise at least one excipient.

Also disclosed are pharmaceutical compositions and dosage forms that comprise at least one compound that can reduce the rate by which at least one active ingredient will decompose. Such compounds are referred to herein as “stabilizers.” Pharmaceutical compositions and dosage forms useful herein can also be prepared using methods disclosed in U.S. 2009/0143404 A1, U.S. 2006/0025422 A1, and/or using conventional methods.

Like the amounts and types of excipients, the amounts and specific types of active ingredients in a dosage form may differ depending on factors such as, but not limited to, the route by which it is to be administered to patients.

In an embodiment, dosage forms comprise at least one second active ingredient. The specific amount of the at least one second active agent may depend on the specific active agent used, the diseases and/or disorders being treated and/or managed, and the amount(s) of the at least one compound disclosed herein, and any optional additional active agent(s) concurrently administered to the patient.

Disclosed herein are methods relating to the effective use of at least one compound chosen from lurasidone and pharmaceutically acceptable salts, solvates, clathrates and stereoisomers thereof to treat patients with at least one CNS disorder. For example, patients with an initial response to therapy with at least one compound chosen from lurasidone and pharmaceutically acceptable salts, solvates, clathrates and stereoisomers thereof and patients with no response to therapy with at least one compound chosen from lurasidone and pharmaceutically acceptable salts, solvates, clathrates and stereoisomers thereof may be identified following initial treatment with the at least one compound.

Thus, in an embodiment, disclosed herein are methods for treating a patient having at least one CNS disorder with at least one compound chosen from lurasidone and pharmaceutically acceptable salts, solvates, clathrates and stereoisomers thereof by administering a dose of the at least one compound for a first dosage period; identifying early responders of treatment with the at least one compound and non-responders to treatment with the at least one compound; maintaining treatment with the at least one compound for early responders; and increasing the dose of the at least one compound for non-responders; wherein the second dose improves the safety and/or efficacy of the treatment during the second dosage period.

In some embodiments, an “early responder” is a patient who responds to treatment with at least one compound chosen from lurasidone and pharmaceutically acceptable salts, solvates, clathrates and stereoisomers thereof within the first two weeks after initial treatment. In some embodiments, a “non-responder” is a patient who fails to respond to treatment with at least one compound chosen from lurasidone and pharmaceutically acceptable salts, solvates, clathrates and stereoisomers thereof within the first two weeks of initiating treatment. Response to initial treatment may be measured by, for example, an improvement, such as ≥20%, on the Positive and Negative Syndrome Scale (PANSS).

In an embodiment, the first dose is 80 mg per day of at least one compound chosen from lurasidone and pharmaceutically acceptable salts, solvates, clathrates and stereoisomers thereof and the first dosage period is two weeks. In an embodiment, the second dose is 160 mg per day of at least one compound chosen from lurasidone and pharmaceutically acceptable salts, solvates, clathrates and stereoisomers thereof and the second dosage period is four weeks.

In an embodiment, the first dose is 20 mg per day of at least one compound chosen from lurasidone and pharmaceutically acceptable salts, solvates, clathrates and stereoisomers thereof and the first dosage period is seven days. In an embodiment, the second dose is a flexible dose of 20 mg, 40 mg, or 60 mg, of at least one compound chosen from lurasidone and pharmaceutically acceptable salts, solvates, clathrates and stereoisomers thereof and the second dosage period begins on the eighth day.

Yet in another embodiment, the first dose is 40 mg per day or 120 mg per day of at least one compound chosen from lurasidone and pharmaceutically acceptable salts, solvates, clathrates and stereoisomers thereof and the first dosage period is six weeks. In an embodiment, the second dose is a flexible dose of 40 mg to 120 mg of at least one compound chosen from lurasidone and pharmaceutically acceptable salts, solvates, clathrates and stereoisomers thereof and the second dosage period lasts for six months.

In one embodiment, the first dose is 80 mg per day or 160 mg per day of at least one compound chosen from lurasidone and pharmaceutically acceptable salts, solvates, clathrates and stereoisomers thereof and the first dosage period is six weeks. In an embodiment, the second dose is a flexible dose of 40 mg to 120 mg of at least one compound chosen from lurasidone and pharmaceutically acceptable salts, solvates, clathrates and stereoisomers thereof and the second dosage period lasts for twelve months.

In yet another embodiment, the first dose is a flexible dose of 40 mg per day to 120 mg per day of at least one compound chosen from lurasidone and pharmaceutically acceptable salts, solvates, clathrates and stereoisomers thereof and the first dosage period is twelve months. In an embodiment, the second dose is a flexible dose of 40 mg to 120 mg, of at least one compound chosen from lurasidone and pharmaceutically acceptable salts, solvates, clathrates and stereoisomers thereof and the second dosage period lasts for six months.

In at least one embodiment, the first dose is 40 mg per day or 80 mg per day of at least one compound chosen from lurasidone and pharmaceutically acceptable salts, solvates, clathrates and stereoisomers thereof and the first dosage period is seven days. In an embodiment, the second dose is 40 mg per day or 80 mg per day, of at least one compound chosen from lurasidone and pharmaceutically acceptable salts, solvates, clathrates and stereoisomers thereof and the second dosage period lasts for seven days. In another embodiment, the third dose is a flexible dose of 40 mg to 120 mg, of at least one compound chosen from lurasidone and pharmaceutically acceptable salts, solvates, clathrates and stereoisomers thereof and the second dosage period lasts for four weeks.

In an embodiment, disclosed herein are methods for treating a patient having at least one CNS disorder with at least one compound chosen from lurasidone and pharmaceutically acceptable salts, solvates, clathrates and stereoisomers thereof by administering a dose of the at least one compound for a first dosage period; identifying early responders of treatment with the at least one compound and non-responders to treatment with the at least one compound; decreasing the dose of the at least one compound for early responders; and increasing the dose of the at least one compound for non-responders; wherein the subsequent dose improves the safety and/or efficacy of the treatment during the second dosage period.

In an embodiment, the first dose is 80 mg per day of at least one compound chosen from lurasidone and pharmaceutically acceptable salts, solvates, clathrates and stereoisomers thereof and the first dosage period is two weeks. In an embodiment, the second dose is 20 mg per day of at least one compound chosen from lurasidone and pharmaceutically acceptable salts, solvates, clathrates and stereoisomers thereof and the second dosage period is four weeks.

In an embodiment, disclosed herein is a method of treating a patient with at least one CNS disorder with at least one compound chosen from lurasidone and pharmaceutically acceptable salts, solvates, clathrates and stereoisomers thereof, comprising:
administering to a patient at least one compound chosen from lurasidone and pharmaceutically acceptable salts, solvates, clathrates and stereoisomers thereof for a first dosage period;
classifying the patient as an early responder or a non-responder based on a response indicator;
determining a dose and dosage period of the at least one compound based on the response indicator;
administering a second dose of at least one compound chosen from lurasidone and pharmaceutically acceptable salts, solvates, clathrates and stereoisomers thereof to the patient over the second dosage period; wherein the second dose and second dosage period improve the safety and/or efficacy of treatment with the at least one compound.

In another embodiment, disclosed herein is a method of treating a patient with at least one CNS disorder with at least one compound chosen from lurasidone and pharmaceutically acceptable salts, solvates, clathrates and stereoisomers thereof, comprising:
administering to a patient at least one compound chosen from lurasidone and pharmaceutically acceptable salts, solvates, clathrates and stereoisomers thereof for a first dosage period;
determining a second dose and dosage period of the at least one compound based on a response indicator;
administering a second dose of at least one compound chosen from lurasidone and pharmaceutically acceptable salts, solvates, clathrates and stereoisomers thereof to the patient over the second dosage period; wherein the second dose and second dosage period improve the safety and/or efficacy of treatment with the at least one compound.

In one embodiment, the second dose is lower than the first dose. Yet in another embodiment, the second dose is higher than the first dose.

Examples of at least one CNS disorder are disclosed herein elsewhere. In an embodiment, the at least one CNS disorder is bipolar disorder. In an embodiment, the at least one CNS disorder is schizophrenia, cognitive impairment associated with schizophrenia (“CIAS”), and/or another negative symptom associated with schizophrenia. In an embodiment, the at least one CNS disorder is mixed depression.

In an embodiment, the initial dose of the at least one compound is 20 mg. In another embodiment, the first dose of the at least one is 40 mg. In another embodiment, the first dose of the at least one compound is 50 mg. In another embodiment, the first dose of the at least one compound is 60 mg. In another embodiment, the first dose of the at least one compound is 80 mg. In yet another embodiment, the first dose of the at least one compound is 120 mg. In another embodiment, the first dose is a flexible dose ranging from 20 mg to 60 mg, such as from 30 mg to 50 mg, or a flexible dose of 40 mg to 80 mg, such as from 50 mg to 70 mg.

In an embodiment, the first dosage period is 2 days, 5 days, 6 days, 7 days, 8 days, 9 days, 10 days, 14 days, 15 days, 16 days, 20 days, 21 days, 25 days, or 28 days.

In an embodiment, the response indicator is the PANSS score measured after two weeks of the initial dose of at least one compound chosen from lurasidone and pharmaceutically acceptable salts, solvates, clathrates and stereoisomers thereof. The PANSS score is evaluated to determine the second dose and dosage period of treatment with at least one compound chosen from lurasidone and pharmaceutically acceptable salts, solvates, clathrates and stereoisomers thereof.

In an embodiment, a PANSS score of ≥20% will determine whether the patient is an early responder or a non-responder to the initial treatment of at least one compound chosen from lurasidone and pharmaceutically acceptable salts, solvates, clathrates and stereoisomers thereof. In an embodiment, a score of >20% will determine whether the patient qualifies as an early responder or a non-responder.

In another embodiment, the response indicator is improvement in cognition measured after two weeks of the initial dose of the at least one compound. Improvement in cognition is evaluated to determine the second dose and dosage period of treatment with the at least one compound.

In an embodiment, the second dose of at least one compound chosen from lurasidone and pharmaceutically acceptable salts, solvates, clathrates and stereoisomers thereof is 80 mg per day. In another embodiment, the second dose of the at least one compound is 120 mg, 140 mg, 160 mg, 180 mg, 200 mg, 220 mg, 240 mg, 260 mg, 280 mg. In an embodiment, the second dose of the at least one compound ranges from 100 mg to 300 mg. In another embodiment, the second dose is a flexible dose ranging from 40 mg to 160 mg, or a flexible dose of 60 mg to 180 mg, or a flexible dose of 80 mg to 250 mg.

In an embodiment, the second dosage period is 2 to 6 weeks, for example 4 weeks. In another embodiment, the second dosage period is longer than 6 weeks, such as 12 weeks. In yet another embodiment, the second dosage period is 24 weeks, 2 months, 3 months, 4 months, 6 months, or until improvement is observed.

In one embodiment, at least one compound chosen from lurasidone and pharmaceutically acceptable salts, solvates, clathrates and stereoisomers thereof can be administered at an amount of about 40 mg per day, in a single or divided doses. In an embodiment, a second dose of at least one compound chosen from lurasidone and pharmaceutically acceptable salts, solvates, clathrates and stereoisomers thereof can be administered at an amount of about 80 mg per day, in a single or divided doses. In an embodiment, at least one compound chosen from lurasidone and pharmaceutically acceptable salts, solvates, clathrates and stereoisomers thereof can be administered at an amount of about 120 mg per day, in a single or divided doses. In an embodiment, a second dose of at least one compound chosen from lurasidone and pharmaceutically acceptable salts, solvates, clathrates and stereoisomers thereof can be administered at an amount of about 160 mg per day, in a single or divided doses. In an embodiment, a second dose of at least one compound chosen from lurasidone and pharmaceutically acceptable salts, solvates, clathrates and stereoisomers thereof can be administered at an amount of about 60 mg per day, in a single or divided doses.

In some embodiments, the methods disclosed herein can additionally be used to track or perform quality control on human research trials or to monitor the patient response to a drug regimen by providing a means to confirm that the patient is responding or not responding to specific treatments. These methods can be used in connection with, for example, the management of patient treatment, clinical trials, and cell-based research.

In an embodiment, the methods disclosed herein can be used to track patient response during individual treatment regimes, or during clinical trials. For example, a response indicator, such as the PANSS score, can be assessed after initiating treatment with lurasidone during a clinical trial to determine whether patients are responding or not responding to the initial dose and/or dosage period.

Accordingly, assessing the response to an initial dose and/or dosage period of at least one compound chosen from lurasidone and pharmaceutically acceptable salts, solvates, clathrates and stereoisomers thereof, such as a PANSS score, can provide useful information as to whether the patients would be responsive to the treatment by at least one compound chosen from lurasidone and pharmaceutically acceptable salts, solvates, clathrates and stereoisomers thereof, or whether the patients may respond to an increased dose and/or dosage period of treatment with the at least one compound. Tracking patient response to therapy with at least one compound chosen from lurasidone and pharmaceutically acceptable salts, solvates, clathrates and stereoisomers thereof may improve the efficacy of treatment and subsequent reduction in psychiatric symptoms.

In an embodiment, disclosed herein are methods of treating an early responder of therapy with at least one compound chosen from lurasidone and pharmaceutically acceptable salts, solvates, clathrates and stereoisomers thereof in a patient having at least one CNS disorder, comprising:
administering at least one compound chosen from lurasidone and pharmaceutically acceptable salts, solvates, clathrates and stereoisomers thereof to the patient over a first dosage period;
assessing the response of the patient to the first dosage and dosage period; and
administering a lower dose of the at least one compound chosen from lurasidone and pharmaceutically acceptable salts, solvates, clathrates and stereoisomers thereof to the patient over a second dosage period;
wherein the second dose and second dosage period improve the safety and/or efficacy of treatment with the at least one compound.

In an embodiment, disclosed herein are methods of treating an early responder of therapy with at least one compound chosen from lurasidone and pharmaceutically acceptable salts, solvates, clathrates and stereoisomers thereof in a patient having at least one CNS disorder, comprising:
administering at least one compound chosen from lurasidone and pharmaceutically acceptable salts, solvates, clathrates and stereoisomers thereof to the patient over a first dosage period;
assessing the response of the patient to the first dosage and dosage period; and
administering the same dose of at least one compound chosen from lurasidone and pharmaceutically acceptable salts, solvates, clathrates and stereoisomers thereof as the initial dose to the patient over a second dosage period;
wherein the second dose and second dosage period improve the safety and/or efficacy of treatment with at least one compound chosen from lurasidone and pharmaceutically acceptable salts, solvates, clathrates and stereoisomers thereof.

In an embodiment, disclosed herein are methods of treating a non-responder of therapy with at least one compound chosen from lurasidone and pharmaceutically acceptable salts, solvates, clathrates and stereoisomers thereof in a patient having at least one CNS disorder, comprising:
administering at least one compound chosen from lurasidone and pharmaceutically acceptable salts, solvates, clathrates and stereoisomers thereof to the patient over a first dosage period;
assessing the response of the patient to the first dosage and dosage period; and
administering a higher dose of at least one compound chosen from lurasidone and pharmaceutically acceptable salts, solvates, clathrates and stereoisomers thereof to the patient over a second dosage period;
   wherein the second dose and second dosage period improve the safety and/or efficacy of treatment with at least one compound chosen from lurasidone and pharmaceutically acceptable salts, solvates, clathrates and stereoisomers thereof.

In another embodiment, disclosed herein are methods of treating a non-responder of therapy with at least one compound chosen from lurasidone and pharmaceutically acceptable salts, solvates, clathrates and stereoisomers thereof in a patient having at least one CNS disorder, comprising:
administering at least one compound chosen from lurasidone and pharmaceutically acceptable salts, solvates, clathrates and stereoisomers thereof to the patient over a first dosage period;
assessing the response of the patient to the first dosage and dosage period; and
administering a higher dose of at least one compound chosen from lurasidone and pharmaceutically acceptable salts, solvates, clathrates and stereoisomers thereof to the patient over a second dosage period;
assessing the response of the patient to the second dosage and dosage period; and
administering a third dose of at least one compound chosen from lurasidone and pharmaceutically acceptable salts, solvates, clathrates and stereoisomers thereof to the patient over a third dosage period,
wherein the third dose and third dosage period improve the safety and/or efficacy of treatment with at least one compound chosen from lurasidone and pharmaceutically acceptable salts, solvates, clathrates and stereoisomers thereof.

In an embodiment, the third dose is an amount ranging from 120 mg to 500 mg. In an embodiment, the third dosage period is 3 weeks, 1 year, or until improvement is observed.

Examples of at least one CNS disorder are disclosed herein elsewhere. In an embodiment, the at least one CNS disorder is bipolar disorder. In another embodiment, the at least one CNS disorder is schizophrenia, cognitive impairment associated with schizophrenia, and/or another negative symptom associated with schizophrenia. In another embodiment, the at least one CNS disorder is mixed depression.

In an embodiment, the initial dose of at least one compound chosen from lurasidone and pharmaceutically acceptable salts, solvates, clathrates and stereoisomers thereof is 40 mg, 80 mg, 120 mg, or 160 mg. In another embodiment, the first dose of at least one compound chosen from lurasidone and pharmaceutically acceptable salts, solvates, clathrates and stereoisomers thereof is 20 mg, 40 mg, 60 mg, 80 mg, or 120 mg. In another embodiment, the first dose is a flexible dose ranging from 20 mg to 60 mg, such as from 30 mg to 50 mg, or a flexible dose of 40 mg to 80 mg, such as from 50 mg to 70 mg.

In an embodiment, the first dosage period is 2 days, 5 days, 6 days, 7 days, 8 days, 9 days, 10 days, 14 days, 15 days, 16 days, 20 days, 21 days, 25 days, or 28 days.

In an embodiment, the response indicator is the PANSS score measured after two weeks of the initial dose of at least one compound chosen from lurasidone and pharmaceutically acceptable salts, solvates, clathrates and stereoisomers thereof. The PANSS score is evaluated to determine the second dose and dosage period of treatment with at least one compound chosen from lurasidone and pharmaceutically acceptable salts, solvates, clathrates and stereoisomers thereof.

In an embodiment, a PANSS score of ≥20% will determine whether the patient is an early responder or a non-responder to an initial treatment of at least one compound chosen from lurasidone and pharmaceutically acceptable salts, solvates, clathrates and stereoisomers thereof. In some embodiments, a score of >20% will determine whether the patient qualifies as an early responder or a non-responder.

In another embodiment, the response indicator is improvement in cognition measured after two weeks of the initial dose of at least one compound chosen from lurasidone and pharmaceutically acceptable salts, solvates, clathrates and stereoisomers thereof. Improvement in cognition is evaluated to determine the second dose and dosage period of treatment with at least one compound chosen from lurasidone and pharmaceutically acceptable salts, solvates, clathrates and stereoisomers thereof.

In an embodiment, the second dose of at least one compound chosen from lurasidone and pharmaceutically acceptable salts, solvates, clathrates and stereoisomers thereof is 80 mg per day. In another embodiment, the second dose of the at least one compound is 120 mg, 140 mg, 160 mg, 180 mg, 200 mg, 220 mg, 240 mg, 260 mg, 280 mg. In yet another embodiment, the second dose of the at least one compound ranges from 100 mg to 300 mg. In another embodiment, the second dose is a flexible dose ranging from 40 mg to 160 mg, or a flexible dose ranging from 60 mg to 180 mg, or a flexible dose ranging from 80 mg to 250 mg.

In an embodiment, the second dosage period is 2 to 6 weeks, for example 4 weeks. In another embodiment, the second dosage period is longer than 6 weeks, such as 12 weeks. In yet another embodiment, the second dosage period is 24 weeks, 2 months, 3 months, 4 months, 6 months, or until improvement is observed.

In one embodiment, the third dose is an amount ranging from 120 mg to 500 mg. In another embodiment, the third dosage period is 3 weeks, 1 year, or until improvement is observed.

In an embodiment, at least one compound chosen from lurasidone and pharmaceutically acceptable salts, solvates, clathrates and stereoisomers thereof can be administered at an amount of about 40 mg per day, in a single or divided doses. In an embodiment, a second dose of at least one compound chosen from lurasidone and pharmaceutically acceptable salts, solvates, clathrates and stereoisomers thereof can be administered at an amount of about 80 mg per day, in a single or divided doses. In an embodiment, at least one compound chosen from lurasidone and pharmaceutically acceptable salts, solvates, clathrates and stereoisomers thereof can be administered at an amount of about 120 mg per day, in a single or divided doses. In an embodiment, a second dose of at least one compound chosen from lurasidone and pharmaceutically acceptable salts, solvates, clathrates and stereoisomers thereof can be administered at an amount of about 160 mg per day, in a single or divided doses. In an embodiment, a second dose of at least one compound chosen from lurasidone and pharmaceutically acceptable salts, solvates, clathrates and stereoisomers thereof can be administered at an amount of about 60 mg per day, in a single or divided doses.

A kit for treatment and/or prevention of at least one CNS disorder comprising at least two compartments, and further comprising

in a first compartment, at least one compound chosen from lurasidone and a pharmaceutically acceptable salt thereof in a first dosage amount, and
in a second compartment, at least one compound chosen from lurasidone and a pharmaceutically acceptable salt thereof in a second dosage amount,
wherein the first dosage amount is administered to a patient in need thereof for a first dosage period,
the patient is classified as an early responder or a non-responder based on a response indicator, and
the second dosage amount is administered to the patient for a second dosage period based on the response indicator, and wherein the second dose and second dosage period improve the safety and/or efficacy of treatment with at least one compound chosen from lurasidone and a pharmaceutically acceptable salt thereof.

Also provided herein are kits for treatment and/or prevention of at least one CNS disorder comprising at least two compartments, a first compartment and a second compartment, wherein the first compartment contains lurasidone or a pharmaceutically acceptable salt thereof in a first dosage amount, and the second compartment contains lurasidone or a pharmaceutically acceptable salt thereof in a second dosage amount.

Also provided herein are kits for treatment and/or prevention of at least one CNS disorder comprising at least two compartments, and further comprising

in a first compartment, at least one compound chosen from lurasidone and a pharmaceutically acceptable salt thereof in a first dosage amount, and
in a second compartment, at least one compound chosen from lurasidone and a pharmaceutically acceptable salt thereof in a second dosage amount,
wherein the first dosage amount is administered to a patient in need thereof for a first dosage period, and
a second dosage amount is administered to the patient for a second dosage period based on a response indicator, and
wherein the second dose and second dosage period improve the safety and/or efficacy of treatment with at least one compound chosen from lurasidone and a pharmaceutically acceptable salt thereof.

Certain embodiments disclosed herein can be illustrated by the following examples, which are not intended to limit the full extent of the disclosure herein in any way.

### EXAMPLES

### I. Quality of Well-Being

Quality of Well-Being (QWB) was measured according to the procedures well-established in the art. The QWB was a self administered questionnaire (QWB-SA), where the combined score ranged from 0 to 1.0 (death to optimal functioning). A patient diagnosed with schizophrenia answered five sections assessing the presence/absence of 19 chronic symptoms or problems, physical symptoms, and mental health symptoms and behaviors as well as an assessment of a person's mobility, physical activity and social activity including completion of role expectations.

QWB-SA assessments were conducted at Baseline and after a 6-week double-blind treatment period in patients treated with 80 mg or 160 mg of lurasidone per day. In addition, the relationship between other indicators of therapy outcome, for example, cognitive composite scores, PANSS scores, Negative Symptom Assessment Scale scores (NSA), and Montgomery-Asberg Depression Rating Scale scores (MADRS), was evaluated in relation to quality of life, as assessed by the QWB-SA. An endpoint ANCOVA analysis showed significant improvement in the QWB-SA score compared to placebo for both 80 mg and 160 mg lurasidone groups, as shown in FIG. 1. The correlation between quality of life and other outcome measures are shown in FIG. 2.

Significant improvements in health-related quality of life were found in patients who had received 80 mg and 160 mg of lurasidone over a 6-week, placebo controlled study. In addition, partial correlations were found between improvement of QWB-SA and PANSS scores, QWB-SA and MADRS scores, QWB-SA and NSA scores, and QWB-SA and cognitive composite scores.

### II. Metabolic Effects

Metabolic effects were analyzed in patients undergoing lurasidone therapy for the treatment of schizophrenia over a period of 1 year. Lurasidone was administered to patients at an initial dose of 40 mg per day, and adjusted up to a maximum of 120 mg/day over a 16-week period, followed by a fixed dose regimen from week-16 to week-52. Changes in weight, total cholesterol, triglycerides, fasting glucose, and prolactin were measured and compared to baseline levels.

As shown in FIG. 3, there were no adverse changes in mean weight, lipids, and prolactin from baseline levels after the 1-year study in patients with schizophrenia.

In addition to no adverse weight gain or increase in lipid or prolactin levels, lurasidone therapy was associated with a gradual and sustained improvement in total PANSS and Brief Psychiatric Rating Scale (BPRS) scores in patients with schizophrenia over a 1-year period.

In another study, potential adverse effects of lurasidone therapy (dose range, 20-120 mg) in patients with schizophrenia was evaluated in a short-term (6-week) study and long-term (6-month and 12-month) study, and compared to patients treated with olanzapine 15 mg), haloperidol (10 mg), risperidone (4 mg), quetiapine XR (600 mg), and placebo. Table 1 below summarizes the baseline metabolic parameters of the patients in the short-term study.

TABLE 1
Baseline Characteristics of Short-term Safety Sample

| | | | OLANZ | RISP | | |
|---|---|---|---|---|---|---|
| | LUR 20-160 mg | HAL 10 mg | 15 mg | 4 mg | QXR 600 mg | Placebo |
| Mean values | (N = 1508) | (N = 72) | (N = 122) | (N = 65) | (N = 119) | (N = 708) |
| Weight, kg | 77.05 | 87.75 | 76.01 | 61.98 | 72.14 | 77.95 |
| BMI, kg/m² | 26.50 | 28.61 | 26.01 | 23.01 | 25.47 | 26.78 |
| Obese (BMI≥30 kg/m²), % | 25.20% | 37.50% | 25.41% | 6.15% | 19.33% | 26.41% |
| Glucose, mg/dL | 96.8 | 97.4 | 94.1 | 92.6 | 93.2 | 97.3 |
| HbA1c, % | 5.57% | 5.64% | 5.58% | 5.38% | 5.54 | 5.57% |
| Total cholesterol, | 191.3 | 199.1 | 193.6 | 180.2 | 182.4 | 191.4 |
| mg/dL | | | | | | |
| Triglycerides, | 147.7 | 182.8 | 133.4 | 117.0 | 137.2 | 151.2 |
| mg/dL | | | | | | |

Sample sizes may vary for individual tests

The mean change in weight and BMI was assessed at the LOCF endpoint for the short-term study. As shown in FIGS. 4-5, treatment with lurasidone was associated with minimal increases in weight and BMI as compared to other treatments. FIG. 6 summarizes the mean change in weight and BMI in observed cases after 6 months and 12 months of treatment

The median change in total cholesterol and triglycerides, and glucose and HbA1c, was also assessed for both the short-term and long-term studies. As shown in FIGS. 4-9, treatment with lurasidone was not associated with disturbances in lipids or glycemic control in both the short-term and long-term studies.

### III. Comparison of Receptor Binding of Lurasidone and Other Antipsychotics

Receptor binding of lurasidone was compared to currently available first and second generation antipsychotics. In vitro binding studies were carried out according to the procedures well-established in the art. Membranes from male Wistar rat brain tissues or cells expressing cloned human receptors were prepared under assay conditions summarized in Ishibashi, T. et al. (Journal of Pharmacology and Experimental Therapeutics (2010)). Functional assays were carried out to analyze the binding affinity and effect on receptor function for dopamine D1-D4 receptor subtypes, serotonin 5-HT_{1A}, 5-HT_{2A}, 5-HT_{2C}, 5-HT₆, 5-HT₇, norepinephrine α₁, α_{2A}, α_{2C}, histamine H₁ receptors, and muscarinic M₁ receptors.

The binding profiles of lurasidone and other currently available antipsychotics are summarized in Table 2. FIG. 10 illustrates the binding affinities of each antipsychotic tested, and FIGS. 11 and 12 demonstrate the effects of lurasidone on receptor function. The high selectivity of lurasidone for the D₂ receptor, serotonin 5-HT_{2A}, 5-HT_{1A} (partial agonist) and 5-HT₇ receptors, moderate affinity for α_{2C} adrenoreceptor, and low or negligible binding affinity for serotonin 5-HT_{2C} receptor, α₁ adrenoreceptor, muscarinic M₁ and histamine H₁ receptors indicate that lurasidone is consistent with low liability for inducing sedation, EPS, cognitive impairment, hypotension and metabolic side effects.

TABLE 2
In vitro Receptor Binding Profiles

| Binding | | | | | | |
|---|---|---|---|---|---|---|
| Affinities | | | | | | |
| (Ki; nM) | Lurasidone | Haloperidol | Risperidone | Aripiprazole | Clozapine | Olanzapine |
| Dopamine: | | | | | | |
| D₁ | 262^{a} | 83 | 60.6 | 387 | 189 | 58 |
| D₂ | 0.994 | 2.0 | 4.9 | 0.95 | 431 | 72 |
| D₃ | 15.7 | 12 | 12.2 | 4.5 | 646 | 63 |
| D₄ | 29.7 | 5.0 | 7.5 | >1000 | 22.3 | 21 |
| Serotonin: | | | | | | |
| 5-HT_{1A} | 6.38 | 1202 | 427 | 5.6 | 105 | 2063 |
| 5-HT_{2A} | 0.47 | 53 | 0.17 | 8.7 | 5.4 | 2.0 |
| 5-HT_{2c} | 415^{b} | 3085 | 12 | 75 | 10.7 | 6.4 |
| 5-HT₆ | — | 3666 | 2241 | 574 | 17 | 6.0 |
| 5-HT₇ | 0.495 | 378 | 6.6 | 10 | 18 | 105 |
| Norepinephrine: | | | | | | |
| α₁ | 47.9^{a} | 12 | 5.0 | 25 | 1.6 | 109 |
| α_{2A} | 40.7 | 1113 | 151 | 74 | 142 | 314 |
| α_{2C} | 10.8 | 550 | 1.3 | 38 | 34 | 29 |
| Histamine H₁ | >1000^{c} | 3002 | 5.184 | 29 | 2.0 | 4.9 |
| Acetylcholine M₁ | >1000 | >10000 | >10000 | 6776 | 14 | 24 |

Cloned cells expressing human receptors unless otherwise noted: ^{a} rat; ^{b} porcine; ^{c} guinea pig Kᵢ for α_{1A} is reported for all antipsychotics except lurasidone

### IV. Lurasidone Monotherapy for the Treatment of Bipolar I Depression: Results of a 6-Week, Double-Blind, Placebo-Controlled Study

A study was conducted to evaluate the efficacy and safety of lurasidone, flexibly dosed at 20 to 60 mg/day or 80 to 120 mg/day compared to placebo, in the treatment of major depressive episodes associated with bipolar I disorder.

The study was designed as shown in the diagram below:

### Dose Titration:

Lurasidone 20-60 mg/day: started at 20 mg/day for 7 days, then flexible dosing
Lurasidone 80-120 mg/day: started at 20 mg/day, then increased by 20 mg/day every 2 days until 80 mg/day, then flexible dosing

### Key Study Entry Criteria:

Adult outpatients, ages 18-75 years, inclusive
DSM-IV-TR diagnosis of Bipolar I disorder with current major depressive episode
   Depressive episode≥4 weeks and <12 months in duration
   MADRS score≥20 at screening and baseline

YMRS≤12 at screening and baseline

Patients meeting the Diagnostic and Statistical Manual of Mental Disorders, 4^{th} Ed., text Revision (DSM-IV-TR) criteria for bipolar I depression, with or without rapid cycling, with a Montgomery Asberg Depression Rating Scale (MADRS) total score≥20 and a Young Mania Rating Scale score≤12, were randomly assigned in a double-blind fashion to treatment with lurasidone 20 to 60 mg/day (LUR20-60), lurasidone 80 to 120 mg/day (LUR80-120), or placebo (PBO) once daily for 6 weeks.

Changes from DB baseline (DB-BL) to week 6 in MADRS and CGI-bipolar severity (CGI-BP-S) depression scores, respectively, were analyzed using mixed model for repeated measures (MMRM). Additional secondary outcome measures, e.g., Sheehan Disability Scale (SDS), were analyzed using analysis of covariance, last observation carried forward (ANCOVA-LOCF), or logistic regression.

The dose titration patterns for this study were as follows:

(1) Lurasidone 20 to 60 mg/day group: started at 20 mg/day for 7 days, then flexible dosing (e.g., 20 mg/day, 40 mg/day, or 60 mg/day);
(2) Lurasidone 80 to 120 mg/day group: started at 20 mg/day then increased by 20 mg/day every 2 days until the dose reached 80 mg/day, then flexible dosing (e.g., Days 1-2: 20 mg/day; Days 3-4: 40 mg/day; Days 5-6; 60 mg/day; Day 7: 80 mg/day; After Day 7, flexible dosing, e.g., 80 mg/day, 100 mg/day, or 120 mg/day).

This study found that monotherapy with lurasidone, flexibly dosed at 20 to 60 mg/day or 80 to 120 mg/day, significantly reduced depressive symptoms in patients with bipolar I depression compared to placebo.

The study found that completion rates were 74.1% in the LUR20-60 group (n/N=123/166; mean modal dose, 34.9 mg/d), 73.4% in the LUR80-120 group (n/N=124/169; mean modal dose, 92.3 mg/d) and 74.7% in the PBO group (n/N=127/170).

As shown in FIG. 13, lurasidone treatment resulted in significantly greater MADRS score reduction at week 6 for both the LUR20-60 group (−15.4; p<0.001; effect size=0.51) and the LUR80-120 group (−15.4; p<0.001, effect size=0.51) as compared to PBO (−10.7). Both LUR groups separated significantly from PBO from week 2 onward.

As shown in FIG. 14, lurasidone treatment resulted in significantly greater reduction in CGI-BP-S depression scores for both the LUR20-60 group (−1.8; p<0.001) and the LUR80-120 group (−1.7; p<0.001) compared to PBO (−1.1). Responder rates (reduction in MADRS total score≥50%) were significantly higher for LUR20-60 (53%) and LUR80-120 (51%) compared to PBO (30%; p<0.001 for both comparisons). Both LUR20-60 and LUR80-120 groups also showed significant improvement over PBO on the Sheehan Disability Scale (SDS) (p≤0.01) (see FIG. 15).

This study also evaluated the safety and tolerability of using 20 to 60 mg/day of lurasidone and 80 to 120 mg/day of lurasidone as compared to placebo. The safety results are provided in Table 3 below.

TABLE 3
Safety and Tolerability

| Weight and | | | Lurasidone | | Lurasidone | |
|---|---|---|---|---|---|---|
| Metabolic | Placebo | | 80-120 mg | | 20-60 mg | |
| (at LOCF endpoint) | N | change* | N | change* | N | change* |
| Weight (kg) mean | 151 | −0.04 | 147 | +0.02 | 143 | +0.56 |
| change | | | | | | |
| Cholesterol (mg/dL) | 147 | −3.0 | 144 | −3.0 | 140 | 0.0 |
| median change | | | | | | |
| Triglycerides (mg/dL) | 147 | +8.0 | 144 | −2.0 | 140 | +3.0 |
| median change | | | | | | |
| Glucose (mg/dL) | 148 | +0.5 | 143 | 0.0 | 140 | −1.0 |
| median change | | | | | | |

| | | | Lurasidone |
|---|---|---|---|
| Adverse Events | Placebo | Lurasidone 20-60 mg | 80-120 mg |
| (incidence ≥5%)** | (n = 168) | (n = 164) | (n = 167) |
| Nausea | 13 (7.7%) | 17 (10.4%) | 29 (17.4%) |
| Headache | 20 (11.9%) | 23 (14.0%) | 15 (9.0%) |
| Akathisia | 4 (2.4%) | 13 (7.9%) | 18 (10.8%) |
| Insomnia | 14 (8.3%) | 8 (4.9%) | 11 (6.6%) |
| Somnolence | 7 (4.2%) | 7 (4.3%) | 11 (6.6%) |
| Sedation | 3 (1.8%) | 5 (3.0%) | 12 (7.2%) |
| Dizziness | 13 (7.7%) | 4 (2.4%) | 10 (6.0%) |

*LOCF-endpoint change (sample size varies due to available data);
**Incidence ≥5% in the combined lurasidone group or the placebo group
Treatment-emergent mania was observed in a similar proportion of subjects treated with lurasidone 20-60 mg (1%), lurasidone 80-120 mg (0%), and placebo (1%). No subjects discontinued from the study due to mania or hypomania
On the Columbia Suicide Severity Rating Scale (C-SSRS), the incidence of “any suicidal ideation or behavior” was similar on lurasidone 20-60 mg/d (6.8%), lurasidone 80-120 mg/d (6.2%), and placebo (7.4%)

The discontinuation rates due to adverse events for LUR20-60 (7%) and LUR80-120 (6%) were similar to PBO (6%). As shown above, for LUR20-60, LUR80-120, and PBO, respectively, the most frequently reported adverse events were nausea (10.4%, 17.4%, 7.7%), headache (14.0%, 9.0%, 11.9%), and akathisia (7.9%, 10.8%, 2.4%). Minimal changes in weight, lipids and measures of glycemic control were observed.

### V. Lurasidone Adjunctive Therapy to Lithium or Divalproex for the Treatment of Bipolar I Depression: Results of a 6-Week, Double-Blind, Placebo-Controlled Study

The primary objective of this study was to evaluate the efficacy and safety of lurasidone (20 to 120 mg/day, flexibly dosed) in combination with lithium (Li) or divalproex (VPA) as compared to placebo (in combination with Li or VPA) for the treatment of patients with major depressive episodes associated with bipolar I disorder (most recent episode depressed with or without rapid cycling disease course (≥4 episodes of mood disturbance but <8 episodes in the previous 12 months), and without psychotic features (diagnosed by Diagnostic and Statistical Manual of Mental Disorders, 4^{th} Ed., text Revision (DSM-IV-TR) criteria) as measured by the Montgomery-Asberg Depression Rating Scale (MADRS) total score). Changes from DB baseline (DB BL) in MADRS total score and secondary efficacy outcomes were analyzed using either mixed model for repeated measures (MMRM) or analysis of covariance, last observation carried forward (ANCOVA-LOCF), or logistic regression.

A secondary objective of this study was to evaluate the efficacy of lurasidone (20 to 120 mg/day, flexibly dosed) in combination with lithium or divalproex as measured by: (1) Global severity assessed by the Clinical Global impression Bipolar Version, Severity of Illness (CGI-BP-S) score (depression), and (2) Subject self-report of functional impairment associated with bipolar depressive symptoms, assessed by the Sheehan Disability Scale (SDS) total score.

Further objectives of this study included evaluation of the following: (1) Subject self-report of overall depressive symptom severity assessed by the Quick Inventory of Depressive Symptomatology-Self Report (QIDS-SR16) total score, (2) Treatment-emergent mania assessed by the Young Mania Rating Scale (YMRS), (3) Anxiety symptoms assessed by the Hamilton Rating Scale for Anxiety (HAM-A), (4) Quality of life and functional impairment assessed by the Quality of Life Enjoyment and Satisfaction Questionnaire-Short Form (Q-LES-Q-SF) scales, (5) Treatment response defined as ≥50% reduction from baseline on the MADRS total score, and (6) Symptom remission defined as a MADRS total score of ≤12 at endpoint.

The study was designed as shown in the diagram below:

### Dose Titration:

Days 1-3: 20 mg/day
Days 4-6: 40 mg/day
Day 7: 60 mg/day

### Key Study Entry Criteria:

Adult outpatients, ages 18-75 years, inclusive
DSM-IV-TR diagnosis of Bipolar I disorder with current major depressive episode
   Depressive episode≥4 weeks and <12 months in duration
   MADRS score≥20 at screening and baseline
   YMRS≤12 at screening and baseline

The study was conducted with approximately 340 subjects (N=170 per treatment group).

Subjects meeting DSM-IV-TR criteria for bipolar I depression with a Montgomery Asberg Depression Rating Scale (MADRS) score≥20 were randomized to 6 weeks of double-blind (DB) treatment with either lurasidone 20-120 mg/day (LUR) or placebo (PBO), both adjunctive to either lithium (Li) or divalproex (VPA). Therapeutic. blood levels of Li or VPA had to be maintained for ≥28 days prior to randomization.

During the study, lurasidone (20 mg/day, 40 mg/day, 60 mg/day, 80 mg/day, 100 mg/day or 120 mg/day) or placebo, in combination with lithium or divalproex was randomly administered to patients, orally, once daily in the evening with a meal or within 30 minutes after eating, for 6 weeks. Lurasidone was initiated at a dose of 20 mg per day for days 1, 2, and 3, followed by 40 mg per day for days 4, 5, and 6, and 60 mg per day for day 7, and was then flexibly dosed for weeks 2 to 6. The total duration of treatment was 6 weeks.

Patients were required to continue treatment with lithium or divalproex during the screening period and throughout the study. Dose adjustments of lithium or divalproex were permitted during the trial to ensure that trough serum levels remained within the protocol-specified range (0.6 mEq/L to 1.2 mEq/L for lithium or 50 mEq/L to 125 μg/mL for divalproex).

Efficacy was evaluated by measuring the change from baseline in MADRS total score for patients treated with lurasidone (20 to 120 mg/day) in combination with lithium or divalproex and comparing the results to the change from baseline in MADRS total score for patients treated with placebo in combination with lithium or divalproex. A mixed model for repeated measures (MMRM) was utilized using the ITT population. The results are depicted in FIG. 16. The MMRM model included treatment, visit, pooled center, stratification variable (lithium or divalproex), baseline MADRS total score, and the treatment-by-visit interactions. An unstructured covariance matrix was used for the within-subject correlation. Missing observations were not imputed for this analysis.

Efficacy was also evaluated by measuring the change from baseline in CGI-BP-S score (depression) and the change from baseline in SDS total score. The results are depicted in FIGS. 17 and 18. CGI-BP-S was analyzed using the MMRM method described above, and SDS was analyzed using an analysis of covariance (ANCOVA) model with treatment, visit, pooled center, stratification variable (lithium or divalproex) and the baseline value of SDS. The ITT population was used for the analyses.

This study found that adjunctive use of lurasidone, compared to placebo, significantly reduced depressive symptoms in patients with bipolar I depression who had inadequate response to at least 4 weeks of monotherapy with lithium or divalproex. Treatment with lurasidone also significantly improved self-rated measures of social, family, and occupational function as well as quality of life.

This study also evaluated the safety and tolerability of using lurasidone in combination with lithium or divalproex. The safety results are provided in Table 4 below. Patients were evaluated fen (1) Adverse effects, discontinuation due to adverse effects (AEs), and serious adverse effects (SAEs), (2) Movement disorders as assessed by Abnormal Involuntary Movement Scale (AIMS), Barnes Akathisia Rating Scale (BARS), and the Simpson-Angus Scale (SAS), and (3) Weight, laboratory measures, vital signs, and electrocardiograms (ECGs).

TABLE 4
Safety and Tolerability

| | Placebo + Li/VPA | Lurasidone + Li/VPA |
|---|---|---|
| | (N = 146-150) | (N = 157-162) |
| | Change* | Change* |
| Weight, kg, mean | +0.14 | +0.23 |
| BMI, kg/m², mean | +0.05 | +0.08 |
| Cholesterol, mg/dL, median | −3.8 | −3.0 |
| Triglycerides, mg/dL, | −4.0 | +4.5 |
| median | | |
| Glucose, mg/dL, median | +1.0 | +1.0 |
| Prolactin, ng/mL, median | 0.0 | +3.8 |

| | | |
|---|---|---|
| | Placebo + Li/VPA | Lurasidone + Li/VPA |
| Adverse Events** | (N = 163) | (N = 183) |

| | | | | |
|---|---|---|---|---|
| Nausea | 18 | (11.0%) | 32 | (17.5%) |
| Headache | 20 | (12.3%) | 19 | (10.4%) |
| Somnolence | 7 | (4.3%) | 16 | (8.7%) |
| Tremor | 7 | (4.3%) | 15 | (8.2%) |
| Akathisia | 7 | (4.3%) | 14 | (7.7%) |
| Insomnia | 9 | (5.5%) | 13 | (7.1%) |
| Diarrhea | 11 | (6.7%) | 8 | (4.4%) |

*LOCF-endpoint change (sample size varies due to available data);
**Incidence ≥5% in the combined lurasidone group or the placebo group
Treatment-emergent hypomania as an adverse event was observed in one subject each in the lurasidone and placebo groups
On the Columbia Suicide Severity Rating Scale (C-SSRS), the incidence of “any suicidal ideation or behavior” was similar on lurasidone + Li/VPA (5.0%) and placebo + Li/VPA (3.1%)

The discontinuation rates due to adverse events were similar in the lurasidone and placebo groups (6% vs. 8%). As shown above, the most frequently reported adverse events for lurasidone were nausea, headache, somnolence, and tremor. Minimal changes in weight, lipids, and measures of glucemic control were observed.

From the foregoing, it will be appreciated that, although specific embodiments have been described herein for the purpose of illustration, various modifications may be made without deviating from the spirit and scope of what is disclosed herein.

1. A method of treating a patient of at least one CNS disorder who did not respond to a first dose of lurasidone and/or its pharmaceutically acceptable salt for a first dosage period, comprising:
administering to the patient a second dose of at least one of lurasidone and a pharmaceutically acceptable salt thereof over a second dosage period,
wherein the second dose is higher than the first dose.

2. The method of claim 1, wherein the at least one CNS disorder is at least one of bipolar disorder, schizophrenia, cognitive impairment associated with schizophrenia, a negative symptom associated with schizophrenia, and mixed depression.

3. The method of claim 1, wherein the first dose is one of 20 mg, 40 mg, 60 mg, 80 mg, and 120 mg, per day.

4. The method of claim 3, wherein the first dose is 80 mg.

5. The method of claim 1, wherein the first dosage period is 1 to 4 weeks.

6. The method of claim 5, wherein the first dosage period is 2 weeks.

7. The method of claim 1, wherein the patient is a non-responder to the first dose for the first dosage period based on a PANSS score.

8. The method of claim 7, wherein the patient is a non-responder to the first dose for the first dosage period based on the PANSS score measured after the first dosage period.

9. The method of claim 8, wherein the patient is a non-responder to the first dose for the first dosage period based on the PANSS score of ≥20%.

10. (canceled)

11. The method of claim 1, wherein the second dose is one of 40 mg, 60 mg, 80 mg, and 120 mg and 160 mg, per day.

12. The method of claim 1, wherein the second dosage period is greater than 6 weeks.

13. The method of claim 12, wherein the second dosage period is 12 weeks.

14. The method of claim 1, wherein the second dosage period is 2 to 6 weeks.

15-17. (canceled)

18. The method of claim 1, wherein the at least one CNS disorder is bipolar disorder.

19. The method of claim 1, wherein the at least one CNS disorder is schizophrenia.
